SEQUENCE LISTING (3 sequences)

SEQ ID NO: 1 - DNA, 29 bp, Leishmania sp.
ggccaatatttaaattggataacttggcg

SEQ ID NO: 2 - DNA, 31 bp, Leishmania sp.
ccggaatatttaaatatcggtgaactttcgg

SEQ ID NO: 3 - DNA, 8493 bp, Leishmania sp.
aaattggataacttggcgaaacgccaagctaatacatgaaccaaccgggtgttctccactccagacggtgggcaaccatcgtcgtgagacgcccagcgaatgaatgacagtaaaaccaatgccttcactggcagtaacacccagcagtgttgactcaattcattccgtgcgaaagccggcttgttccggcgtcttttgacgaacaactgccctatcagctggtgatggccgtgtagtggactgccatggcgttgacgggagcgggggattagggttcgattccggagagggagcctgagaaatagctaccacttctacggagggcagcaggcgcgcaaattgcccaatgtcaaaacaaaacgatgaggcagcgaaaagaaatagagttgtcagtccatttggattgtcatttcaatgggggatatttaaacccatccaatatcgagtaacaattggaggacaagtctggtgccagcacccgcggtaattccagctccaaaagcgtatattaatgctgttgctgttaaagggttcgtagttgaactgtgggctgtgcaggtttgttcctggtcgtcccgtccatgtcggatttggtgacccaggcccttgcagcccgtgaacattcaaagaaacaagaaacacgggagtggttcctttcctgatttacgcatgtcatgcatgccagggggcgtccgtgattttttactgtgactaaagaagcgtgactaaagcagtcatttgacttgaattagaaagcatgggataacaaaggagcagcctctaggctaccgtttcggcttttgttggttttaaaggtctattggagattatggagctgtgcgacaagtgctttcccatcgcaacttcggttcggtgtgtggcgcctttgaggggtttagtgcgtccggtgcgatagggagaccacaacggtttccctctagtgcgtgaagggttaccgcaacgatgcgcaatggactcccccgctttccatttcgtcaccttccgcctctctctctctctctctcaccatctacgcgtgcacatcatcaactgtctcttgtcggtgctcaccaccctcaaccacccctcactttcaaggcttcccgaacgcacacaaaaggcgtgaaaaccgctcgcgtgtgttgagccgtccaccgtagccctccccctgtccccgggggatccactagttctagaggatcggaggtgtgtgtgcccttgtgtgctgtgtgtgggtggacgcagcgatgcccggcgcgtgtgggcacctccttgggtgcgcgcccgccgtggcagctgcgcgtgcgtgcgagatgtgaggcagaggaagaggaaggcgatgcgggcgacacgcagaggtgcggcggacgtaggggggaaatggacgagcaggcgcgctgtgaatcggagctgcggcaccacccaagtcgtggtgccccgcgaatggctgttctgccgccctcgcttcacgcctccccctcccctcgcgtgccctcgcgtggcctcccttgttatccctctctctcgcacgcacacggatacgcgagcccgctattctgccttcgtctggctctttgtattctgcttgcttcttcagcacacttgtgtgctgtgcgttcagcgatatcttccactactttgttttctcctccccctcgggaggtgcttcgcttgtgctttgacggtggtgcgtggctgctgggtcatgtgccgggcgtgcgcgcctccgccgcctccctgcagcttgtgggtctggctgcgttcgcaccgcgctcgcgtgcatgcacatgcctgcactgcgtcgggaacgacttccgggcgcgttggccccccgcctctgcagccacggtctgtttattgattgtgcttgcttcatcggctcttctctgcgcgcgtgcgtgcgtgcgtgtgcgtgtccgtgcgtatgcgtgaggcgcaacggtccccagagcaaggcatgtcgaggggaacactatagacgcatgtgtacgtgtacacgatgtgtatacgtatacgtgtaccgaatggtgcgtgcgcgtgtgcagcattgccgtgacggcatgtacgaagcgctgcagtgggatggaccctgtgcgcgtgccggagaggtagtgtcgcgtgtgggtgcggagtgatggaggctagggggcttacgagcaccgtcgcttttcccccgatggcggctggcacgcagcgcacgcaccggggatgtgtgacgtgcgtcctgtgcgcctctccctctccccttgtcgccggcgcatggatgcaccgctgttgtgtgaggttgcccgcacctgcgttgttgcctgtgatgacgtccctccctctcttgcactctccccgtccccacctgccctgcaccgtggtcgactgctcccgacgccctgcacagactctcgtcgccaccaccagcagcagccctctatatacccgccactgccgtagcgttcgggccgtggctctgcgtttcacttgctctcccctcgctctgttcattgcttccttctgttcccctcgctgcccgcgtccggagatctatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgacccggatctccctttagtgagggttaattagtcctgcattaatgaatcggccaacgcgcggggagaggcggtttgcgtattgggcgctcttccgctactcgggtgtcgcacacactgtaaaacgcccccgccggctctgtcacgcaagaaacgagagcaaaaagaccggtagactatatcacgcacaatcaccgcgtgtgcgtctccctgggtgaagacacccatcgcacccttcgacagccgcccttatgcctattcaccgtctgtagaacacacaagaggaatagcccggtgccgcgtgcaagactgcggcttctgcacgcactatgctcgtttccgcctctctctctttgtgcgcgtgtgtgtgtgtgtgtcggagtggccctcccgttacgtcttttgggggtgggtgatagcggcagatgctgcttcgaccttgtgcgccgcaccggtgccgttggctacactgcggaaggcaacacagaacacaccctgtgccatttcttcttttttttttgctttcacccaccttttccccgtgcttccccatctttccccctctttccctaacgtacattgcacctctccttatcgtgcagtcacacgctaccactcaacgctccctgcaacactggagtgagtcgctagaaataattttgtttaactttaagaaggagatatacatagtgaccggatcctagtatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaactagcctcggagatccactagttctagttctagggggcgcgaattcagatcctcgtgtgagcgttcgcggaatcggtcgctcgtgtttatgcccgtcttggtgttgtgctcgcaaggcggtgcagcaggataccgtcgccctcctctctccttgcttctctgttcttcaattcgcgatctcacagaggccggctgtgcacgcccttcctcaccctccttttcccacctctcggccaccggtcggctccgttccgtctgccgtcgagaagggacgggcatgtgcagctcctccctttctctcgcgcgcgcatcttctcttgcttgtggcactcacgctcatgcgtcaaggcggccccacgcgagcccctgcgctcccttccctcttgcgcatccgtagccggactggtcgatgcgcaaggccggcatgaaggagcgcgtgccctcaagagcggactatcatgccctacgtgggccacgcagcgatgaggccggcttcgcggagatgcgtcacgcacgtgccagatgatgccgtacgccttccttgacttgcgcccccctctcttcctccgtctctcactctctctctctcacacacacacacacacacacacacacacaaagctccggttcgtccggccgtaacgccttttcaactcacggcctctaggaatgaaggagggtagttcgggggagaacgtactggggcgtcagaggtgaaattcttagaccgcaccaagacgaactacagcgaaggcattcttcaaggataccttcctcaatcaagaaccaaagtgtggagatcgaagatgattagagaccattgtagtccacactgcaaacgatgacacccatgaattggggatcttatgggccggcctgcggcagggtttaccctgtgtcagcaccgcgcccgcttttaccaacttacgtatcttttctattcggcctttaccggccacccacgggaatatcctcagcacgttttctgttttttcacgcgaaagctttgaggttacagtctcaggggggagtacgttcgcaagagtgaaacttaaagaaattgacggaatggcaccacaagacgtggagcgtgcggtttaatttgactcaacacggggaactttaccagatccggacaggatgaggattgacagattgagtgttctttctcgattccctgaatggtggtgcatggccgcttttggtcggtggagtgatttgtttggttgattccgtcaacggacgagatccaagctgcccagtagaattcagaattgcccatagaatagcaaactcatcggcgggttttacccaacggtgggccgcattcggtcgaattcttctctgcgggattcctttgtaattgcacaaggtgaaattttgggcaacagcaggtctgtgatgctcctcaatgttctgggcgacacgcgcactacaatgtcagtgagaacaagaaaaacgacttttgtcgaacctacttgatcaaaagagtggggaaaccccggaatcacatagacccacttgggaccgaggattgcaattattggtcgcgcaacgaggaatgtctcgtaggcgcagctcatcaaactgtgccgattacgtccctgccatttgtacacaccgcccgtcgttgtttccgatgatggtgcaatacaggtgatcggacaggcggtgttttatccgcccgaaagttcaccgatatttaaatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggattt

(19) United States
(12) Patent Application Publication
(10) Pub. No.: US 2001/0010928 A1
(43) Pub. Date: Aug. 2, 2001

(54) PROTOZOAN EXPRESSION SYSTEM

(76) Inventor: STEPHEN M. BEVERLEY, CLAYTON, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/277,513

(22) Filed: Mar. 26, 1999

(51) Int. Cl. A01N 37/18; A61K 38/00; C12Q 1/68; A61K 31/70; A01N 43/04; C12P 21/06; C12N 15/09; C12P 21/02; C12P 21/04; A61K 48/00

(52) U.S. Cl. 435/69.1; 435/320.1; 435/325; 435/243; 435/69.4; 435/69.5; 435/69.51; 435/69.52; 435/69.6; 435/6; 514/2; 514/44; 435/810

(57) ABSTRACT

A method for the high level production of active, properly processed recombinant protein in trans-splicing organisms is disclosed. The method involves the integration of the gene encoding the recombinant protein of interest into a chromosomal locus where it is transcribed under the direction of the rRNA promoter. The gene is also operably linked to intergenic regions allowing the protein to be translated in these organisms. The recombinant organisms expressing a therapeutic protein can also be used to treat a disease or undesirable condition which is characterized by a deficiency in that protein.

This invention was made with Government support under National Institutes of Health Grant No. AI29646. The Government has certain rights in the invention.

### BACKGROUND OF THE INVENTION

1. Field of the Invention

The present invention generally relates to the production of recombinant proteins in heterologous hosts. More particularly, the invention relates to the production of active, properly processed recombinant proteins in high yields in transgenic protozoan hosts. The invention is useful for the production of purified proteins as well as for the treatment of disease or undesirable conditions.

2. Description of Related Art

An expression system for producing recombinant proteins should have the following characteristics: (1) the ability to easily, inexpensively, and rapidly produce the protein of interest; (2) the ability to produce the protein at high yield; (3) the ability to produce active protein, especially when activity of the protein depends on proper post-translational processing such as glycosylation, acylation, phosphorylation, peptide cleavage, etc.; and (4) the ability to allow the protein to be easily isolated and purified, while retaining biological activity. Several host systems have been developed to achieve these goals.

Prokaryotic expression systems using organisms such as E. coli and Bacillus spp. allow for easy, inexpensive and rapid production of recombinant heterologous proteins. However, these systems are often unable to post-translationally process proteins from eukaryotic sources correctly, which often precludes the production of active protein.

Several eukaryotic systems are also available for the production of recombinant proteins. Yeast and other fungi, mammalian cells, plants and plant cells, and insects and insect cells are examples. For any particular protein one or another of these systems may provide adequate production of active protein. However, there is an ongoing need for alternative systems which may provide advantages for the production of recombinant proteins of interest.

Trans-splicing Eukaryotes

Several genera of eukaryotes, in particular kinetoplastids and other mastigophorid protozoans, process RNA transcripts by trans-splicing (reviewed in Agabian (1990), Cell 61:1157-1160; Graham (1995), Parasitology Today 11:217-223). In this process, an RNA polymerase, usually RNA polymerase II, transcribes most genes into a polycistronic primary transcript which contain intergenic regions encoding a 5′ consensus splice acceptor site 30-70 bases upstream of the translational start site and a 3′ signal for polyadenylation. Introns are not present. RNA processing proceeds by the cleavage and polyadenylation of the primary transcript. A 39 nucleotide spliced leader sequence (SL) from a different transcript is also trans-spliced onto the 5′ end of the translational start site (providing a 5′ cap), creating a mature (capped and polyadenylated) mRNA. Thus, unlike cis-splicing mRNA processing, which occurs in most eukaryotes, the sequence encoding the 5′ cap (here, the SL) is not part of the same primary transcript as the message for the structural gene, but is trans-spliced from a separate transcript.

RNA polymerase I (pol I) normally serves to transcribe ribosomal RNA genes (which are not translated) in eukaryotes. However, in trans-splicing organisms, because primary transcripts of messages to be translated are trans-spliced by a common SL, pol I can serve to transcribe genes which contain a splice acceptor site. Those genes are then polyadenylated, capped with the SL, and translated into proteins. Pol I has been shown to naturally produce transcripts which are translated due to the presence of a splice acceptor site, for example the genes for the variant surface glycoprotein (VSG) and the procyclic acidic repetitive protein (PARP) in Trypanosoma brucei. Production of heterologous genes, mediated by pol I, has also been demonstrated from genes inserted by homologous recombination downstream from the rRNA promoter on the chromosome of T. brucei (Zomerdijk et al. (1991) Nature 353:772-775; Rudenko et al. (1991) EMBO J. 10:3387-3397). However, it has not been previously suggested that the rRNA promoter in trans-spliced organisms can serve to direct the efficient, high level production of recombinant proteins.

Treatment of Disease Caused by Disorders of Cellular Metabolism

A number of diseases are caused by disorders of cellular metabolism. For many of these diseases the nature of the metabolic defect has been identified. For example, Type I diabetes is known to result from defective glucose metabolism associated with decreased levels of insulin. Also, various cancers are believed to result from defective control of cellular division and proliferation associated with mutations in a variety of cellular genes, many of which have been identified. Further, many disorders in cellular metabolism are caused by somatic or hereditary genetic mutations which produce either inappropriate expression of a given gene product or the expression of a defective gene product. Environmental insults such as chemical poisoning, physical damage, or biological infection can also produce defects in cellular metabolism. In addition, cellular aging often results in metabolic disorders.

A common approach to treatment of these diseases consists of systemically administering a pharmaceutical compound or drug that overcomes the metabolic disorder. An example is the administration of exogenous insulin to alleviate the symptoms of Type I diabetes. There are, however, several drawbacks to this type of drug therapy. For a pharmaceutical compound to be effective, it must be administered so that it reaches its site of action at an appropriate concentration. If the compound is provided systemically, e.g., orally or by injection, undesirable side effects may be caused by the presence of systemic levels of the compound required for it to be effective at the site of action. Chemotheraputic agents, for example, often cause such side effects. Drug administration also suffers when potential therapeutic agents are not stable or not readily transportable to the site of action.

For many diseases, the most appropriate therapeutic compound is a specific protein, especially if the disease results from the absence of a function form of the protein. However, delivering any specific protein to its desired site of action can be complicated by its susceptibility to denaturation, proteolytic degradation, and/or poor mobility to its desired site of action.

There is, therefore, a need in the art for effective methods for delivering physiologically useful compounds to a desired site of action in a controlled fashion.

### SUMMARY OF THE INVENTION

Among the several objects of the present invention may be noted the provision of methods and compositions useful for the production of high levels of recombinant protein in trans-splicing eukaryotes. Another object of the invention is the provision of methods and compositions useful for the production of high levels of properly processed, active proteins in trans-splicing organisms. A more specific object of the invention is the provision of a constitutive expression system in Leishmania spp. utilizing the promoter of the Leishmania major rRNA. It is also an object of the invention to provide a eukaryotic system for high level expression of recombinant proteins as an alternative to currently available eukaryotic systems. It is another object of the present invention to provide a means of treating a disease or undesirable condition in an mammal, more particularly a human, by infecting the mammal with a transgenic parasitic kinetoplastid protozoan which produces a protein, when a deficiency of an active form of the protein is the cause of the disease or undesirable condition. It is still another object of the invention to provide methods and compositions for delivering physiologically useful compounds to a desired site of action in a mammal.

Briefly, therefore, the present invention is directed to an expression cassette comprising flanking regions which are homologous to a region of a ribosomal RNA gene from a Leishmania spp., Crithidia spp. or Leptomonas spp.; intergenic regions which contain information required for RNA transcript processing in the organism; and a marker gene operably linked to the intergenic regions which allows selection of individuals of the organism which are transfected with the DNA molecule.

Additionally, the present invention is directed to an expression cassette comprising flanking regions which are homologous to a conserved region of the small subunit ribosomal RNA gene from an organism which undergoes trans-splicing; intergenic regions which contain information required for RNA transcript processing in the organism; and a marker gene operably linked to the intergenic regions which allows selection of individuals of the organism which are transfected with the DNA molecule.

The present invention is also directed to an expression cassette comprising a promoter for a ribosomal RNA gene from an organism which undergoes trans-splicing; flanking sequences which are homologous to a chromosomal region of the organism; intergenic regions which contain information required for RNA transcript processing in the organism; a marker gene operably linked to the intergenic regions which allows selection of individuals of the organism which are transfected with the DNA molecule.

In a further embodiment, the present invention is directed to recombinant plasmids comprising any of the above three expression cassettes, and DNA sequences which allow selection and replication of the vector in E. coli.

In another aspect, the present invention is directed to a host cell of an organism which undergoes trans-splicing which is transformed with any of the above three expression cassettes, wherein the host cell comprises a chromosome.

In a further embodiment, the present invention is directed to a method of producing a protein, comprising (1) obtaining a host cell of an organism which undergoes trans-splicing, where the host cell contains a chromosome and cellular components and is transformed with an expression cassette integrated into the chromosome and having (a) flanking regions which are homologous to a region of a ribosomal RNA gene from a Leishmania spp., Crithidia spp. or Leptomonas spp.; (b) intergenic regions which contain information required for RNA transcript processing in the organism; (d) a marker gene operably linked to the intergenic regions which allows selection of individuals of the organism; and a second gene encoding a protein, wherein the second gene is operably linked to the intergenic regions, and (2) culturing the host cell under conditions and for a time sufficient to produce the protein.

The present invention is also directed to a method of producing a protein, comprising: (1) obtaining a host cell of an organism which undergoes trans-splicing, where the host cell contains a chromosome and cellular components and is transformed with an expression cassette integrated into the chromosome and having (a) flanking regions which are homologous to a conserved region of the small subunit ribosomal RNA gene from an organism which undergoes trans-splicing; (b) intergenic regions which contain information required for RNA transcript processing in the organism; (c) a marker gene operably linked to the intergenic regions which allows selection of individuals of the organism which are transfected with the DNA molecule; and (d) a second gene encoding a protein, wherein the second gene is operably linked to the intergenic regions, and (2) culturing the host cell under conditions and for a time sufficient to produce the protein.

The present invention is still further directed to a method of producing a protein, comprising: (1) obtaining a host cell of an organism which undergoes trans-splicing, where the host cell contains a chromosome and cellular components and is transformed with an expression cassette integrated into the chromosome and having (a) a promoter for a ribosomal RNA gene from an organism which undergoes trans-splicing; (b) flanking sequences which are homologous to a chromosomal region of the organism; (c) intergenic regions which contain information required for RNA transcript processing in the organism; (d) a marker gene operably linked to the intergenic regions which allows selection of individuals of the organism which are transfected with the DNA molecule; and (e) a second gene encoding a protein, wherein the second gene is operably linked to the intergenic regions, and (2) culturing the host cell under conditions and for a time sufficient to produce the protein.

In another aspect, the present invention is directed to a method for studying virulence or pathogenicity in a trans-splicing organism, comprising infecting an experimental animal with a recombinant host cell, where the host cell contains a chromosome and cellular components and is transformed with an expression cassette integrated into the chromosome and having (a) flanking regions which are homologous to a region of a ribosomal RNA gene from a Leishmania spp., Crithidia spp. or Leptomonas spp.; (b) intergenic regions which contain information required for RNA transcript processing in the organism; (d) a marker gene operably linked to the intergenic regions which allows selection of individuals of the organism; and a second gene encoding a green fluorescent protein, wherein the second gene is operably linked to the intergenic regions.

Additionally, the present invention is directed to a method for studying virulence or pathogenicity in a trans-splicing organism, comprising infecting an experimental animal with a recombinant host cell, where the host cell contains a chromosome and cellular components and is transformed with an expression cassette integrated into the chromosome and having (a) flanking regions which are homologous to a conserved region of the small subunit ribosomal RNA gene from an organism which undergoes trans-splicing; (b) intergenic regions which contain information required for RNA transcript processing in the organism; (c) a marker gene operably linked to the intergenic regions which allows selection of individuals of the organism which are transfected with the DNA molecule; and (d) a second gene encoding a green fluorescent protein, wherein the second gene is operably linked to the intergenic regions.

The present invention is also directed to a method for studying virulence or pathogenicity in a trans-splicing organism, comprising infecting an experimental animal with a recombinant host cell, where the host cell contains a chromosome and cellular components and is transformed with an expression cassette integrated into the chromosome and having (a) a promoter for a ribosomal RNA gene from an organism which undergoes trans-splicing; (b) flanking sequences which are homologous to a chromosomal region of the organism; (c) intergenic regions which contain information required for RNA transcript processing in the organism; (d) a marker gene operably linked to the intergenic regions which allows selection of individuals of the organism which are transfected with the DNA molecule; and (e) a second gene encoding a green fluorescent protein, wherein the second gene is operably linked to the intergenic regions.

In a further embodiment, the present invention is directed to a method of treating a disease or undesirable condition in a mammal, comprising infecting the mammal with an infectious strain of a recombinant host cell, where the host cell contains a chromosome and cellular components and is transformed with an expression cassette integrated into the chromosome and having (a) flanking regions which are homologous to a region of a ribosomal RNA gene from a Leishmania spp., Crithidia spp. or Leptomonas spp.; (b) intergenic regions which contain information required for RNA transcript processing in the organism; (d) a marker gene operably linked to the intergenic regions which allows selection of individuals of the organism; and a second gene encoding a protein which is useful for treating the disease or undesirable condition, and wherein the second gene is operably linked to the intergenic regions.

The present invention is also directed to a method of treating a disease or undesirable condition in a mammal, comprising infecting the mammal with an infectious strain of a recombinant host cell, where the host cell contains a chromosome and cellular components and is transformed with an expression cassette integrated into the chromosome and having (a) flanking regions which are homologous to a conserved region of the small subunit ribosomal RNA gene from an organism which undergoes trans-splicing; (b) intergenic regions which contain information required for RNA transcript processing in the organism; (c) a marker gene operably linked to the intergenic regions which allows selection of individuals of the organism which are transfected with the DNA molecule; and (d) a second gene encoding a protein which is useful for treating the disease or undesirable condition, and wherein the second gene is operably linked to the intergenic regions.

The present invention is still further directed to a method of treating a disease or undesirable condition in a mammal, comprising infecting the mammal with an infectious strain of a recombinant host cell, where the host cell contains a chromosome and cellular components and is transformed with an expression cassette integrated into the chromosome and having (a) a promoter for a ribosomal RNA gene from an organism which undergoes trans-splicing; (b) flanking sequences which are homologous to a chromosomal region of the organism; (c) intergenic regions which contain information required for RNA transcript processing in the organism; (d) a marker gene operably linked to the intergenic regions which allows selection of individuals of the organism which are transfected with the DNA molecule; and (e) a second gene encoding a protein useful for treating the disease or undesirable condition, and wherein the second gene is operably linked to the intergenic regions.

In a further aspect, the present invention is directed to a method of delivering a therapeutic protein to a desired site in a mammal, comprising (a) selecting a trans-splicing organism which is capable of infecting the mammal and residing at the desired site; (b) transfecting the trans-splicing organism with an expression cassette comprising flanking regions which are homologous to a region of a ribosomal RNA gene from a Leishmania spp., Crithidia spp. or Leptomonas spp.; intergenic regions which contain information required for RNA transcript processing in the organism; a marker gene operably linked to the intergenic regions which allows selection of individuals of the organism which are transfected with the DNA molecule; and a second gene encoding the therapeutic protein, wherein the second gene is operably linked to the intergenic regions; and (c) infecting the mammal with the transfected trans-splicing organism.

The present invention is further directed to a method of delivering a therapeutic protein to a desired site in a mammal, comprising (a) selecting a trans-splicing organism which is capable of infecting the mammal and residing at the desired site; (b) transfecting the trans-splicing organism with an expression cassette comprising flanking regions which are homologous to a conserved region of the small subunit ribosomal RNA gene from an organism which undergoes trans-splicing; intergenic regions which contain information required for RNA transcript processing in the organism; a marker gene operably linked to the intergenic regions which allows selection of individuals of the organism which are transfected with the DNA molecule; and a second gene encoding the therapeutic protein, wherein the second gene is operably linked to the intergenic regions; and (c) infecting the mammal with the transfected trans-splicing organism.

The present invention is still further directed to a method of delivering a therapeutic protein to a desired site in a mammal, comprising (a) selecting a trans-splicing organism which is capable of infecting the mammal and residing at the desired site; (b) transfecting the trans-splicing organism with an expression cassette comprising a promoter for a ribosomal RNA gene from an organism which undergoes trans-splicing; flanking sequences which are homologous to a chromosomal region of the organism; intergenic regions which contain information required for RNA transcript processing in the organism; a marker gene operably linked to the intergenic regions which allows selection of individuals of the organism which are transfected with the DNA molecule; and a second gene encoding the therapeutic protein, wherein the second gene is operably linked to the intergenic regions; and (c) infecting the mammal with the transfected trans-splicing organism.

In yet another aspect, the present invention is directed to kits for producing a recombinant protein, comprising any of the above three recombinant plasmids, a living cell of the organism, and instructions.

In still another aspect, the present invention is directed toward the use of the above disclosed expression cassettes, plasmids, and host cells for the treatment of disease and for delivering a therapeutic protein to a desired site in a mammal.

### BRIEF DESCRIPTION OF THE DRAWINGS

FIG. 1. pIR-SAT. Intergenic regions are shown as shaded bars; protein coding regions are represented by arrows, and important restriction sites are shown. Insertion site a is the unique SmaI restriction site at the top of the figure; Insertion site b is the unique BglII restriction site at the upper right of the figure. The nucleotide sequence is provided herein as SEQ ID NO:3.

FIG. 2. Schematic representation of the cloning procedure employed to obtain integrative expression cassettes targeting the small subunit ribosomal DNA of Leishmania spp. The expression plasmids generated are shown. Intergenic regions are shown as open bars, protein coding regions are represented by arrows, and important restriction sites are shown.

FIG. 3. Integration of GFP expression cassettes into the SSU rDNA locus of Leishmania species.

a. Scheme of the targeting approach. The upper bar represents the SwaI fragment excised from pIR1SAT-GFPb. The various intergenic regions are named and drawn in gray. Protein coding regions are shown as labeled arrows; unlabeled arrows represent the SSU indicating the direction of transcription. The lower bar illustrates one genomic copy of the rSSU locus. Important restriction sites are indicated. The two bars are not drawn in scale.

b-e. Southern hybridization analysis of NdeI digested genomic DNA from wild-type (wt) and recombinant L. major Friedlin V1 (b and c) or L. donovani (d and e) harbouring the expression cassettes IR1SAT-GFPa or IR1SAT-GFPb. The filters were either probed with the GFP gene (b and d) or a species specific single copy gene also present in the expression cassette as indicated (c and e).

FIG. 4. Relative intensities of fluorescence generated by L. major Friedlin V1 wild-type (top panel), and the recombinant strains pXG-GFP (middle panel), and SSU::IR1SAT-GFPb (bottom panel).

FIG. 5. Green fluorescence profile, at times indicated, of metacyclic L. major Friedlin V1 wild-type and the recombinant strains SSU::IR1SAT-GFPa and SSU::IR1SAT-GFPb.

FIG. 6. Time course of GFP expression during in vitro cultivation of L. major Friedlin Vi SSU::IR1SAT-GFPa (open symbols) and SSU::IR1SAT-GFPb (closed symbols). Metacyclic promastigotes were inoculated at 1×10⁴ cells/ml and cell density (squares) as well as peak fluorescence of the cells (triangles) were measured daily.

FIG. 7. Stage-specific GFP expression. Promastigotes of wild-type L. major Friedlin V1 or the transgenic cell lines containing SSU::IR1SAT-GFPa and SSU::IR1SAT-GFPb at their 6th day of stationary phase, after PNA agglutination. The fluorescence profile of both the agglutinated and unagglutinated fractions are shown, as well as the fluorescence of lesion derived amastigotes from the same cell lines.

FIG. 8. Microscopic images of an isolated mouse peritoneal macrophage infected with L. major Friedlin V1 SSU::IR1SAT-GFPa. a) Phase contrast image. b) green fluorescence of GFP expressing parasites.

### DETAILED DESCRIPTION OF THE INVENTION

The contents of each of the references cited herein are herein incorporated by reference.

Summary of Abbreviations

The listed abbreviations, as used herein, are defined as follows:

Abbreviations:

FACS=fluorescence-activated cell sorter

GFP=green fluorescent protein

IR=intergenic region

PNA=peanut agglutinin

SAT=Streptothricin acetyl transferase

SSU=small subunit if the ribosomal RNA gene.

A “trypanosomid” refers to a member of the family Trypanosomatidae, which includes the genera Trypanosoma, Leishmania, Crrithidia, and Leptomonas.

“Recombinant protein” refers herein to protein produced through translation of a gene on an expression cassette.

“Expression cassette” refers herein to a piece of DNA produced by recombinant methods which can be transfected into an organism to express a recombinant protein encoded thereon.

Organisms which contain a stably maintained expression cassette are herein referred to as “transfected”, “recombinant”, “transformed” or “transgenic”. The expression cassette is inserted into the target organism by the process of “transfection” or “transformation”.

“Target organism” refers herein to an organism which is to be transformed with an expression cassette.

The term “high yield” refers to the production of a large amount of recombinant protein by a transgenic organism. This amount is generally greater than 1% of total protein produced by the organism. Preferably, the amount is greater than 2% of total protein; most preferably, the amount is greater than 5% of total protein.

The procedures disclosed herein which involve the molecular manipulation of nucleic acids are known to those skilled in the art. See generally Fredrick M. Ausubel et al. (1995), “Short Protocols in Molecular Biology”, John Wiley and Sons, and Joseph Sambrook et al. (1989), “Molecular Cloning, A Laboratory Manual”, second ed., Cold Spring Harbor Laboratory Press, which are both incorporated by reference.

An expression system is provided in which recombinant proteins are produced at high levels in a trans-splicing target organism. This system utilizes a linear expression cassette with (a) regions on both ends of the DNA molecule which are homologous to a chromosomal locus, preferably within the ribosomal RNA (rRNA) gene cluster of the target organism, allowing homologous integration into the organism's chromosome (preferably within the rRNA gene cluster); (b) intergenic regions which contain the information required for directing RNA transcript processing (i.e. trans-splicing and polyadenylation) in the target organism; (c) a marker gene, operably linked to intergenic regions, which allows selection of individuals of the target organism which are stably transfected with the expression cassette; and (d) a gene encoding the protein of interest, operably linked to flanking intergenic regions such that the transcript of the gene is properly processed and subsequently translated into the protein of interest when the DNA molecule is integrated into a rRNA gene of the target organism. When the expression cassette is not directed to the rRNA gene cluster, a promoter must be included on the expression cassette which directs pol I transcription of the gene encoding the protein of interest.

It is to be understood that the expression cassettes, plasmids, and host cells disclosed herein can be used for the treatment of disease and for the delivery of a therapeutic protein to a desired site in a mammal.

This expression system may be utilized with any species which undergoes trans-splicing, including (but not limited to) members of the genera Trypanosoma, Leishmania, Leptomonas, Crithidia, and Caenorhabditis. When the recombinant organism is used for production of the protein of interest in culture, preferred organisms are those which can multiply rapidly in inexpensive media without serum, for example Crithidia spp., Leptomonas spp., and Leishmania tarentolae.

Trans-splicing organisms have several characteristics which make them useful for the production of a recombinant protein of interest using the instant invention. Like bacterial protein production systems, they can grow in culture rapidly and to a high density at room temperature and without added carbon dioxide, and they can be plated on solid media at limiting dilutions to readily pick out rapidly growing colonies arising from single cells, giving them an advantage over mammalian cells. Additionally, the preferred organisms Crithidia spp., Leptomonas spp., and Leishmania tarentolae, can be grown on inexpensive media without serum, providing another advantage over mammalian systems. These organisms also do not have a cell wall, which allows for easier purification of a non-secreted protein than bacteria or fungi.

An additional important advantage in using trans-splicing organisms for producing recombinant proteins is their ability to provide proper post-translational processing of recombinant proteins. In particular, the core glycosylation of recombinant mammalian proteins generally closely resembles that of mammals with little other modifications. The secretory system (i.e. the processing of proteins destined for secretory pathways, including proteins destined for release into the media, targeted to the cell surface, or targeted to a subcellular compartment such as the golgi or endoplasmic reticulum) is also typical of other eukaryotes, including mammals, in that it possesses enzymatic machinery for proper folding and assembly of excreted proteins.

When the recombinant organism is used to infect a mammal to treat a disease or an undesirable condition, preferred species are those which will infect the organism in such a way as to deliver the recombinant protein to a location in the organism where the recombinant protein is therapeutic. Since this method depends on infection of the mammal with the recombinant organism, preferred isolates of these organisms are ones which cause minimal deleterious effects on the mammal and ones which can be eliminated from the mammal when the therapy is no longer desired. Examples of such species are members of the genera Trypanosoma and Leishmania which are pathogenic to mammals. The species to be utilized is selected based on the ability of the candidate species to reside in the host in such a way as to allow delivery of the therapeutic protein to a site where it can be advantageously utilized. For example, in the treatment of a lysosomal storage disease, the pathogen L. major may be selected because it resides in lysosomes, and would thus deliver the therapeutic protein where needed.

In the genus Leishmania, several species cause visceral disease and reside intracellularly, e.g., in lymph nodes, liver, spleen, and bone marrow. Other species of Leishmania cause cutaneous and mucocutaneous diseases and reside intracellularly and extracellularly in skin and mucous membranes of the host mammal. Non-limiting examples are L. major, L. tropica, L. aeithiopica, L. entrietti, L. mexicana, L. amazonesis, L. donovani, L. chagasi, L. infantum, L. braziliensis, L. pananaensis, and L. guyanensis. In the genus Trypanosoma, various species are known to reside in visera, myocardium, or brain of the host, and may also reside in blood, lymph nodes, or cerebrospinal fluid at certain stages of their development. Non-limiting examples are T. cruzi and T. brucei.

The transgenic organisms of the instant invention have certain advantages over other organisms or drug therapy for the treatment of various disease. These organisms can be grown in culture as a saprophyte, unlike viruses, which require host cells for multiplication. As discussed above, they can also be utilized as a self-contained system, since various strains only infect particular cell types or cause a localized infection. These transgenic organisms can thus reliably produce therapeutic proteins at the site where the protein is needed, avoiding side effects or denaturation problems. Since the organisms have the ability to evade their host's immune defense, the delivery of the therapeutic protein can be sustained over an extended period of time.

High level expression of the recombinant protein of interest in this system depends on the utilization of a promoter for a pol I transcribed gene, preferably the promoter to the rRNA gene cluster, to direct the transcription of the protein of interest along with the transcription of the native pol I transcribed gene. The rRNA promoter is preferably utilized by directing the integration of the expression cassette containing the gene for the protein of interest into the endogenous rRNA gene cluster of the target organism. Under this scheme, the gene for the protein of interest is transcribed along with the rRNA gene. Since there are many copies of the rRNA gene in trans-splicing organisms (e.g. more than 160 copies are present in Leishmania donovani [Leon et al. (1978), Nucl. Acids Res. 5:491-504]), the insertion of the expression cassette into one or even several of the endogenous rRNA genes does not appreciably affect the production of the rRNA required for normal growth and metabolism of the transfected organism.

The quantity of a recombinant protein produced by this method is generally at least about two times the quantity of the same protein produced by analogous methods utilizing an episomal vector. Preferably, the method will produce at least about three times the recombinant protein produced using episomal methods; more preferably, at least about five times the amount of recombinant protein will be produced. Most preferably, the present method will produce at least about ten times the amount of recombinant protein as that produced using episomal methods.

An alternative method for utilizing a pol I promoter for transcribing the gene of interest is by including the pol I promoter in the expression cassette, upstream from the gene encoding the protein of interest. When a pol I promoter is so included, the expression cassette may be directed to integrate into any region of the genome of the target organism which would not fatally disrupt normal cellular functions.

The linear expression cassette is directed for integration into a region of the genome (preferably the rRNA gene cluster) of the target organism by including sequences homologous to that region on the ends of the linear expression cassette. The extent to which the transfecting sequences must be complementary to the naturally occurring sequences in order to effect efficient homologous integration of the transfecting sequence can vary. The transfecting sequences must be complementary enough to permit homologous recombination to occur between the transfecting and the endogenous sequence. It is known that the portion of the transfecting sequence closest to the edge of the recombination event is less tolerant of differences than the sequences further away from the edge. The precise length of the flanking sequences can also vary. Flanking sequences about 400 base pairs long or longer are generally effective. The skilled artisan will appreciate these fundamentals and can prepare suitable transfecting sequences using only routine experimentation. Furthermore, only routine experimentation is required to determine the primary nucleotide sequence of the DNA flanking either end of the genetic locus.

When transfected into the target organism, the expression cassette is then integrated into the homologous region of the genome. When the integration is directed to the rRNA gene cluster, a preferred region is a region which is conserved among other species of the same genus as the target organism if one wishes to utilize the expression cassette in the other species. An example of such a conserved region is the highly conserved region of the small subunit (SSU) rRNA gene of Leishmania (Uliana et al. (1994) J. Euk. Microbiol. 41:324-330), which, if utilized on the ends of the expression cassette, would allow homologous integration into any Leishmania species.

In order to direct the proper processing of the primary transcript into a translatable mRNA, intergenic regions are included in the expression cassette. Those regions encode a splice acceptor site and a signal for polyadenylation of the transcript. The intergenic regions included in the expression cassette must be operably linked to the gene encoding the protein of interest, i.e. the regions must be so situated in relation to the gene encoding the protein of interest that they direct the proper trans-splicing of the SL sequence and polyadenylation of the transcript in order to create a translatable message for the protein of interest. For example, as previously discussed, the splice acceptor site must be 30-70 bases upstream of the translational start site of the gene for the protein of interest.

The intergenic regions are selected from those regions which provide the necessary processing information in the target organism. Among the known intergenic regions, some are effective among several species or genera and others are effective only within a particular species. Nonlimiting examples of intergenic regions which are effective and preferred in Leishmania spp. are DST, CYS2, LPG1, and 1.7K.

The sources of these intergenic regions are indicated in Appendix 1, under “SEQ ID NO:3”.

A marker gene is included on the expression cassette in order to select for target organisms in which the DNA molecule has been integrated into the genome. Any marker known in the art which is effective in the target organism can be utilized. Preferred are markers which allow survival of the recombinant target organisms when the wild-type organisms which did not undergo genomic integration of the expression cassette are killed. The most preferred markers are antibiotic resistance genes. Nonlimiting examples of antibiotic resistance genes are NEO (encoding neomycin phosphotransferase), which confers resistance to the aminoglycoside G418 (see, e.g. LeBowitz et al. (1990) Proc. Natl. Acad. Sci. U.S.A. 87:9736-9740), and SAT (encoding Streptothricin acetyl transferase), which confers resistance to noursethricin.

The linear expression cassette is preferably provided as a part of a circular plasmid which can be multiplied in an organism such as E. coli by methods known in the art. The plasmid preferably contains sequences useful for transformation and selection into the organism, such as the bacterial origin of replication and an ampicillin resistance marker. The plasmid preferably has unique restriction sites on either end of the expression cassette which is utilized to linearize the plasmid and eliminate the sequences which are not part of the expression cassette used for protozoan transfection.

Any gene encoding a protein of interest can be inserted into the expression cassette by any method known in the art. As previously discussed, the gene is inserted into the molecule such that the gene is operably linked to the intergenic regions. Examples of genes which can be usefully inserted are the green fluorescent protein of Aequorea victoria (Ha et al. (1996) Mol. Biochem. Parasitol. 77:57-64), the CSP protein of Plasmodium falciparum, γ-interferon, and interleukin 12. Properly post-translationally processed and active recombinant forms of the latter three proteins have been expressed in Leishmania major which were transfected with episomal vectors comprising those genes.

Where the transgenic organism is used for the therapeutic delivery of a protein in a mammal, treatment of various diseases or undesirable conditions of the mammal may be effected. In this treatment, the trans-splicing organism is first selected based on the site of infection, as previously discussed. The organism is then transformed with the gene for the therapeutic protein such that the gene is integrated into a chromosome of the organism and under the control of an rRNA promoter, by methods discussed above. The mammal is then infected with the transgenic organism, which will, in the course of its infection, produce the recombinant protein at the desired site. Non-limiting examples of proteins for this therapy are insulin, γ-interferon, tissue plasminogen activator, β-interferon, erythropoietin, and Factor VIII. Non-limiting examples of diseases or undesirable conditions which may be treated by this therapy are osteoporosis, diabetes, cancer, severe anemia, short stature, and hemophilia. Since several species of Leishmania reside in lysosomes, the treatment of lysosomal storage diseases, particularly Goucher Disease (caused by a deficiency of glucocerebrosidase) and Fabry Disease (deficiency of α-galactosidase A) are preferred disease targets.

The linear, isolated expression cassette is transfected into the target organism by any method known in the art. Preferably, cells of the target organism, in a form which is readily grown in culture (e.g. the promastigote form of trypanosomids) are grown to late log phase, suspended at high density (e.g. 10⁸/ml) in an electroporation cuvette along with the expression cassette, and electroporated. After electroporation, the cells in which the expression cassette has been integrated into the genome are selected according to the requirements of the selection marker, and transformed colonies are isolated and grown according to methods known in the art. After the initial selection and establishment of a stable transformed isolate, selection may be withdrawn since recombinant organisms which have the expression cassette integrated into the genome do not require continuous selection to maintain production of the recombinant protein of interest. This is in contrast to the continuous selection required for the production of a recombinant protein which is encoded on a vector that is maintained in the cell as an episome.

When the recombinant target organism is used to produce and isolate a protein of interest in vitro, the organism is grown by any appropriate method known in the art. When the target organism is one of the organisms preferred for this purpose (Crithidia spp., Leptomonas spp., and Leishmania tarentolae), the organism is preferably grown in media which is inexpensive and allows rapid growth to high cell densities, such as brain-heart infusion medium, which contains 37 g/L brain-heart infusion and 10 μg/ml hemin.

The following examples illustrate the invention.

### Example 1

### Construction of a Universal Integrative Expression System for Leishmania and its Use in Expressing a Heterologous Protein Gene

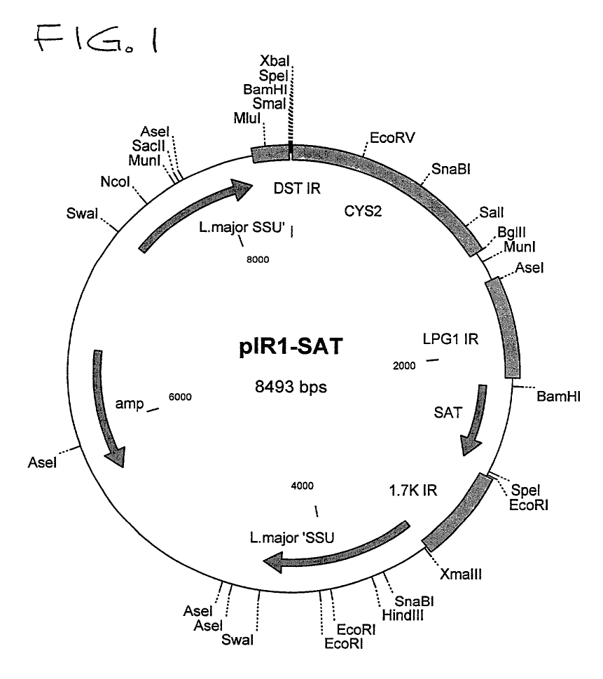

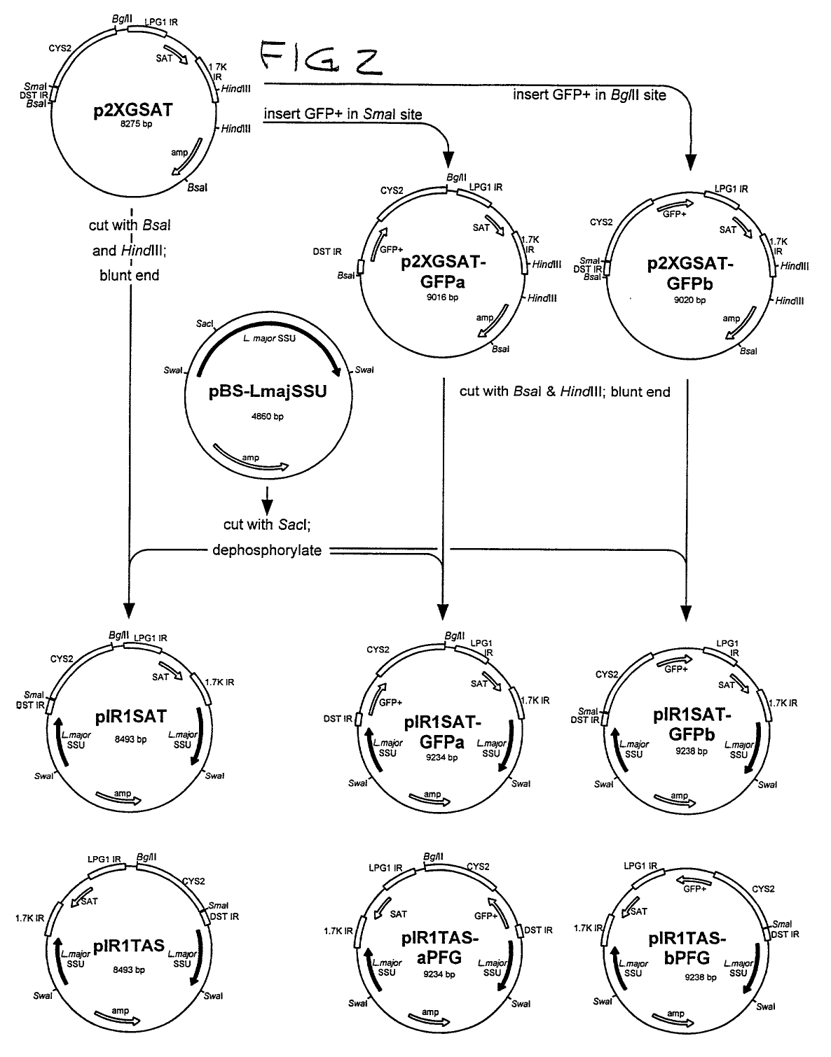

This example describes the construction of (a) a plasmid (pIR1-SAT) (FIG. 1) for integrative expression of proteins in Leishmania spp., (b) an analogous plasmid (p2XGSAT) (FIG. 2) for episomal expression, and (c) the incorporation of GFP into two sites of each plasmid. A variant of the GFP gene, termed GFP+, is utilized in these experiments. This variant is engineered to have enhanced fluorescence and to eliminate codons which are rarely used by Leishmania (Ha et al. (1996) Mol. Biochem. Parasitol. 77:57-64).

The conserved region of the small subunit ribosomal DNA (Uliana et al. (1994) J. Euk. Microbiol. 41:324-330) was amplified from Leishmania major genomic DNA using oligonucleotide primers SMB600 (5′-ggccaatatttaaattggataacttggcg-3′) (SEQ ID NO:1) and SMB601 (5′-ccggaatatttaaatatcggtgaactttcgg-3′) (SEQ ID NO:2) which add SwaI restriction sites (underlined) to either side of the amplification product. The amplified L. major SSU rRNA gene was ligated between the T4 DNA polymerase-treated KpnI and SstI restriction sites of PBSIIKS—(Stratagene). The resulting plasmid was named pBS-LmajSSU (Schwarz, J., unpublished data; Lab strain # B3348) (FIG. 2).

The plasmid p2XGSAT contains the SAT marker flanked by the LPG1 (5′) and 1.7K (3′) intergenic regions, along with DST and CYS2 intergenic regions to be operably linked to a gene for a protein of interest. This plasmid serves as an episomal expression vector in Leishmania spp. The GFP+ gene was excised from plasmid pBS-GFP+ by a HindIII/XbaI double digest and ligated either into the SmaI site or BglII site of p2XGSAT after its treatment with T4 DNA polymerase if necessary. The obtained plasmids were designated p2XGSAT-GFPa or p2XGSAT-GFPb respectively.

The 4.2 kb BsaI/HindIII fragment of p2XGSAT or the respective 4.9 kb fragments of its derivatives p2XGSAT-GFPa or p2XGSAT-GFPb were integrated into the unique SacI site within the SSU of pBS-LmajSSU after removal of single stranded DNA overhangs by T4 DNA polymerase. This non-directional cloning gave six different plasmids with genes either unidirectional with the transcriptional orientation within the ribosomal locus or in the opposite orientation. These expression plasmids were designated as pIR1—series (FIG. 2). Expression cassettes were gel purified after excision from these plasmids by a single SwaI digest.

### Example 2

### Transfection of Leishmania spp.

The Leishmania major strains Friedlin V1 (MHOM/IL/80/Friedlin), Lv39c5 (MRHO/SU/59/P), FEBNI (MHOM/IL/81/FEBNI) and V121 were used as well as the L. donovani strain Ld4. The parasites were grown in supplemented M199 medium and transfections were carried out as described in Kapler et al. (1990) Mol. Cell. Biol. 10:1084-1094. Clonal cell lines were obtained by plating transfected Leishmania on M199 agar plates supplemented with 50-75 μg/ml Nourseothricin (Hans-Knöll-Institut für Naturstoff-Forschung, Jena, Germany).

Metacyclic promastigotes were isolated from cultures at their 6th day of stationary phase by PNA agglutination as described by da Silva and Sacks (1987) Infect. Immun. 55:2802-2806.

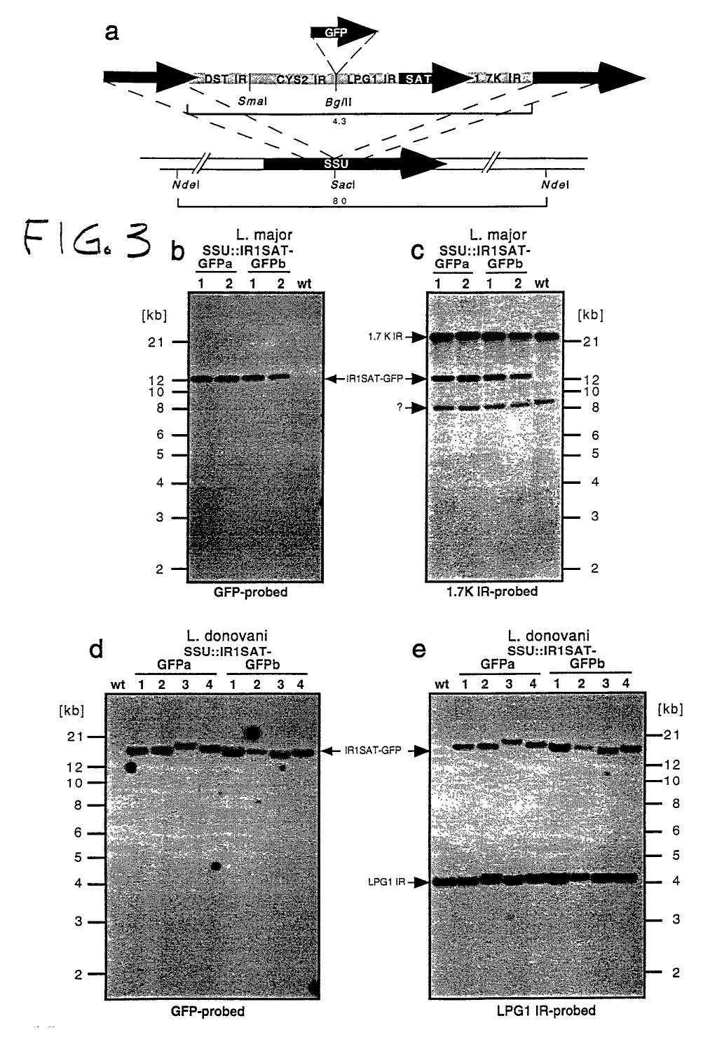

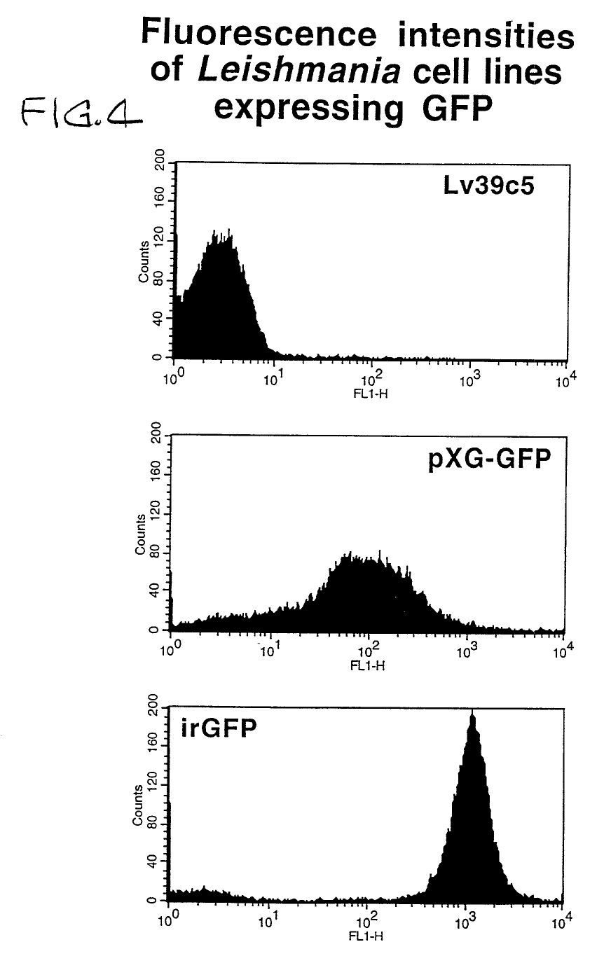

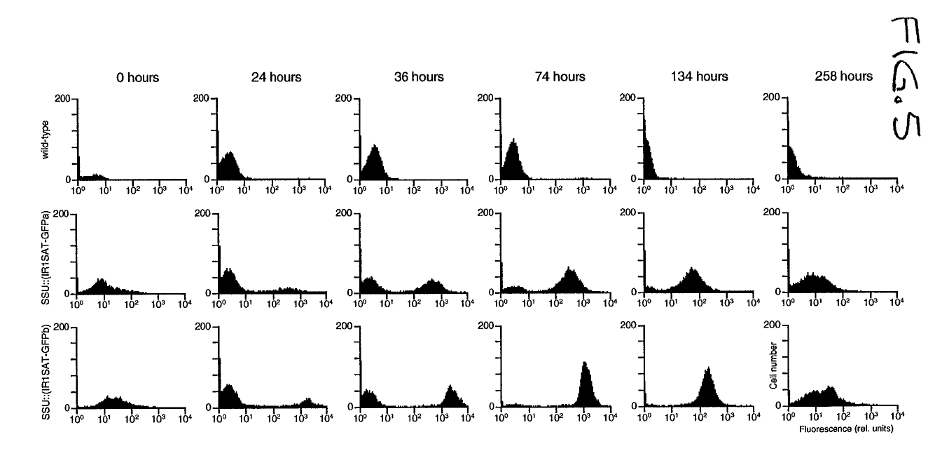

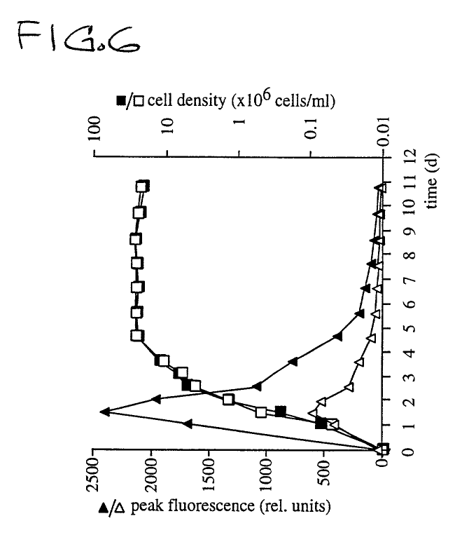

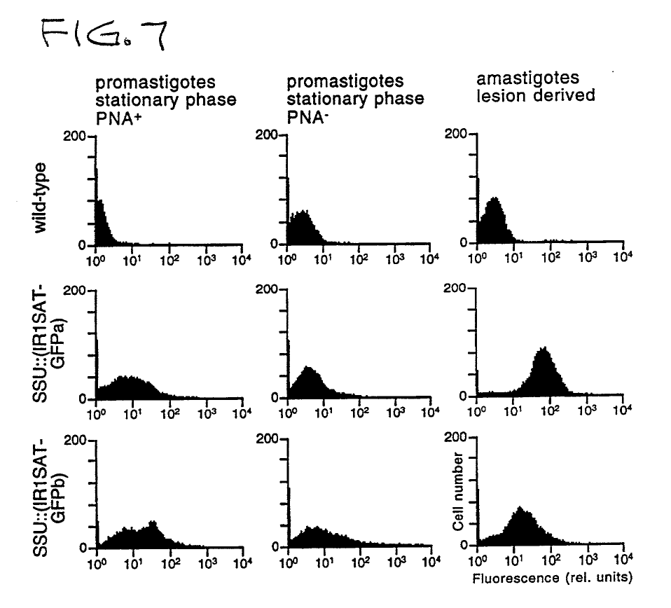

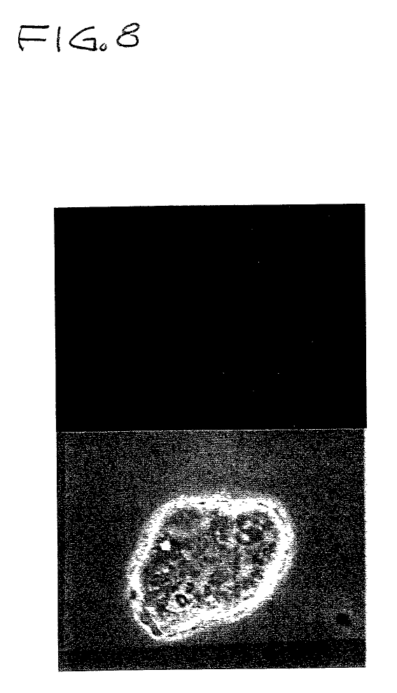

To determine whether the expression cassette was correctly integrated into the SSU rDNA of L. major or L. donovani, Nde I-digested genomic DNA of nourseothricin-resistant clonal cell lines was subjected to Southern blot analyses and the filters were hybridized with the GFP gene as probe (FIG. 3b, d). Genomic DNA of wildtype Leishmania does not hybridize with the GFP gene.

In recombinant L. major strains, 11 kb NdeI fragments hybridize with the GFP gene (FIG. 3b) as expected, because in wild type L. major an 8 kb NdeI fragment harbors the SSU gene (data not shown) whose size is increased by approx. 3 kb in the recombinant locus. A similar result was observed with L. donovani (FIG. 3d), despite the fact that NdeI fragments harboring their SSU are larger and of heterogeneous size. This reflects the different size of recombinant SSU loci in the various L. donovani lines examined. These data indicate that the expression cassette is properly integrated into the SSU rDNA locus. Only a single clone out of 48 clonal cell lines of different L. major strains and L. donovani lines did not have the expression cassette integrated. Such a low proportion of false positive clones illustrates the reliability of the targeting strategy and demonstrates its universal use.

To determine the number of integration events that occurred in each cell line, the Southern blots of NdeI-digested genomic DNA were reprobed with a species-specific single copy gene also present on our expression cassettes. The filter with L. major DNA probed with the 1.7 K IR displays an approx. 22 kb fragment present in all cell lines (FIG. 3c). These fragments represent the endogenous alleles of the 1.7 K IR. Recombinant cell lines also show the 11 kb fragments of the altered SSU rDNA locus. In addition, we observed bands of 8 kb in every L. major cell line. These fragments are of unknown identity but they are most likely unaltered copies of the SSU rDNA, since the template for our 1.7 K IR probe was isolated from pIR1SAT. Minor contamination of this preparation with the SSU rDNA from the plasmid results in a signal of high intensity due to the high copy number of the ribosomal loci. The L. donovani blot was hybridized with the LPG1 IR. This probe hybridized with the two allels present in the genome on a 4.1 kb NdeI fragment. In recombinant L. donovani, the probe also hybridized with bands of the same size as seen with the GFP-probed filter (FIG. 3e). Signal intensities of these filters were quantified using a phosphoimager and revealed that the signals derived from the wild-type allels were twice as strong as the signals obtained from the recombinant SSU locus (data not shown). Thus, only single integration events took place in the examined cell lines.

### Example 3

### Expression of Heterologous Protein in Cultured, Transgenic Leishmania s5D.

Fluorescent activities of Leishmania cell lines were quantified using a Becton Dikinson FACScan. Dead cells were excluded from the analysis. Cell death is determined by their staining with propidium iodine as adapted from Jackson et al. (1984) Science 225:435-438. Briefly, propidium iodine (Sigma) was added to the cell cultures to be examined at a final concentration of 3 μg/ml a few minutes prior to their analysis and red fluorescent cell were not taken into account.

The measurement of fluorescence emitted by recombinant promastigote Leishmania was evaluated. The green fluorescence was first measured during logarithmic proliferation phase, i.e. at cell densities of 5-8×10⁶ cells/ ml. For comparison, green fluorescence was also measured in cell lines transfected with the various expression plasmids generated during the cloning process, as well as pXG-GFP+ (Ha et al. (1996) Mol. Biochem. Parasitol. 77:57-64). Comparisons with the latter plasmid provide a measure of prior art expression levels. FIG. 4 shows the relative fluorescence intensities of a wild-type strain (top panel), a strain transformed with an episomal vector expressing GFP+ (middle panel), and a strain transformed with an integrative vector expressing GFP+. Intensity of fluorescence is measured along the X-axis. The strain expressing GFP+ from the integrative vector is expressing about ten times the recombinant protein (as measured by fluorescence intensity) as the strain expressing GFP+ from an episomal vector (FIG. 4). The peak fluorescence of various cell lines are also listed in Table 1. Untransfected Leishmania display a peak fluorescence of 2 to 15 relative units. This background fluorescence is slightly higher in L. donovani than in L. major for unknown reasons. Parasites transfected with the episomal vector pXG-GFP+ show a peak fluorescence of around 45 relative units. Parasites transfected with expression plasmids containing the GFP gene within expression site b , i.e. p2XGSAT-GFPb or pIR1SAT-GFPb, display a brighter fluorescence than pXG-GFP+ transfected Leishmania. The latter cell line show higher fluorescence activities than the cells harboring expression plasmids with the GFP gene in the expression site a. The presence or absence of conserved ribosomal sequences does not have any impact on the fluorescence emitted by transfected parasites and thus does not affect GFP expression. Among the pIR1—series, two antisense constructs were generated (pIR1TAS-aPFG and pIR1TAS-bPFG—FIG. 2). Those plasmids contained the whole expression cassette, (consisting of the various intergenic regions, the SAT gene as selectable marker and the GFP gene) oriented in antisense to the ribosomal sequences. The fluorescence intensities derived from these plasmids transformed as two episomes (by not linearizing the plasmid before transfection) does not differ significantly from those of their respective sense constructs. As expected, we were not able to obtain cell lines having these two particular expression cassettes integrated.

These fluorescence analyses represent relative production of the green fluorescent protein by the cells transformed with the various expression vectors. It is understood by those skilled in the art that the results obtained with other proteins may differ somewhat, however, similar relative results can be expected. As an example, construct similar to pXG-GFP+, but using the E. coli β-galactosidase gene rather than the green fluorescent protein gene as the heterologous protein yielded about 1% of total protein as heterologous protein (LeBowitz et al. (1990) Proc. Natl. Acad. Sci. U.S.A. 87:9736-9740). The relative yield of β-galactosidase in the pIR1-SAT vector would be expected to be considerably higher.

TABLE 1
Fluorescence intensities of Leishmania cell lines.
The numbers represent the peak fluorescence generated by
promastigotes expressing GFP from various constructs of each
cell line at their mid log phase of proliferation.

| Cell line | Construct | Fluorescence | Intensity |
|---|---|---|---|
| L. major | — | | 2 |
| Lv39c5 | pXG-GFP+ | 47 | |
| | p2XGSAT-GFPa | 12 | |
| | p2XGSAT-GFPb | 73 | |
| | pIR1SAT-GFPa | 15 | |
| | pIR1TAS-aPFG | 12 | |
| | pIR1SAT-GFPb | 99 | |
| | pIR1TAS-bPFG | 140 | |
| | SSU: : (IR1SAT-GFPa) | 161 | |
| | SSU: : (IR1SAT-GFPb) | 963 | |
| L. major | SSU: : (IR1SAT-GFPa) | 222 | |
| Friedlin V1 | SSU: : (IR1SAT-GFPb) | 1041 | |
| L. major | SSU: : (IR1SAT-GFPb) | 1131 | |
| FEBNI | | | |
| V121 | SSU: : (IR1SAT-GFPb) | 943 | |
| L. donovani | — | 15 | |
| Ld4 | pXG-GFP+ | 43 | |
| | SSU: : (IR1SAT-GFPa) | 678 | |
| | SSU: : (IR1SAT-GFPb) | 1563 | |

Expression of GFP from episomes and integrated expression cassettes

The fluorescence of recombinant Leishmania expressing GFP+ increases dramatically upon integration of the expression cassettes into the SSU of the ribosomal locus. Although only a single copy of the GFP gene is integrated, fluorescence of the recombinant Leishmania analyzed rises to approximately 1,000 relative units if the GFP gene is present in expression site b (FIG. 4). This increase in GFP expression is due to the activity of the ribosomal RNA promoter which is located approx. 1 kb upstream of each SSU rRNA gene. This promoter drives transcription of the ribosomal subunits (Uliana et al. (1996) Mol. Biochem. Parasitol. 76:245-255; Gay et al. (1997) Mol. Biochem. Parasitol. 77:193-200). As previously shown with the episomal expression constructs, the GFP gene in expression site b also give a 2 to 5 fold higher fluorescence than the GFP gene in expression site a with the integrated expression cassettes. The different untranslated regions flanking the GFP gene in our expression cassettes account for the differences in expression efficiency of the two expression sites available in our cassette. This is expected, since it is known that intergenic regions have different intrinsic efficiencies.

Developmental regulation of GFP expression

During its life cycle Leishmania undergoes distinct, well defined developmental maturations. In order to study the behavior of our integrative expression system in different stages, the life cycle of Leishmania was mimicked in vitro and the fluorescence of our recombinant cell lines at different developmental stages was measured. First, metacyclic promastigotes were isolated from culture, and inoculated at low density in fresh medium. Growth and GFP expression were followed during cultivation. FIG. 5 shows fluorescence profiles of three selected L. major cell lines at different time points during their in vitro cultivation and illustrates changes in GFP expression. Metacyclic promastigotes did not display fluorescence activity. As the cells entered early logarithmic phase of proliferation their fluorescence increased rapidly to the maximum level at 5-7×10⁵ cells/ml as shown in FIG. 6. The fluorescence decreases at increasing cell densities, even though the cells are still in logarithmic phase. A similar effect has been observed with the yeast Saccharomyces cerevisiae (Ju and Warner (1994) Yeast 10:151-157). The fluorescence returns to almost background levels as the culture reaches stationary phase. Despite the absolute levels of expression the time course of GFP activity is identical in cells harboring the GFP gene in expression site a as in cells with their GFP gene in expression site b. The time course of GFP expression follows transcriptional activity within the ribosomal locus, as is also seen in other organisms (Jacob (1995) Biochem. J. 306:617-626).

Promastigotes resistant to PNA agglutination are considered to be metacyclic cells which are in the infective stage and have stopped dividing (da Silva and Sacks (1987) Infect. Immun. 55:2802-2806). To determine the expression of recombinant GFP at this stage, promastigote Leishmania at their 6th day of stationary phase were subjected to agglutination with PNA. PNA positive and PNA negative cells of wildtype Leishmania and the strains SSU::IR1SAT-GFPa and SSU::IR1SAT-GFPb were analyzed by FACS. PNA+ or procyclic late stationary phase promastigotes and metacyclic promastigotes do not differ in their fluorescence intensities as shown in FIG. 7 and Table 2. While brightness of the SSU::IR1SAT-GFPa strain is hardly above background, members of the SSU::IR1SAT-GFPb strain display a weak fluorescence.

TABLE 2
Stage-dependent GFP expression
The peak fluorescence of L. major Friedlin V1 wild-type
parasites as well as SSU: : IR1SAT-GFPa and SSU: : IR1SAT-GFPb are
displayed.

| | | SSU: : (IR1SAT- | SSU: : (IR1SAT- |
|---|---|---|---|
| | wild-type | GFPa) | GFPb) |
| log phase | | | |
| promastigotes | 4 | 222 | 1041 |
| stationary phase | | | |
| promastigotes PNA+ | 1 | 9 | 32 |
| promastigotes PNA− | 3 | 6 | 27 |
| lesion-derived | 4 | 72 | 37 |
| amastigotes | | | |

### Example 4

### Expression of Heterologous Protein in Leishmania spp. in Infected MacroPhages and Hosts

Fluorescence microscopic investigation of macrophage infection in vitro

The green fluorescence of the transgenic cell lines expressing GFP+ described in previous examples was evaluated in the amastigote stage present in mammalina hosts.

Peritoneal macrophages were isolated from Balb/c mice 2 days after stimulation with sterile starch as described by Behin et al. (1979) Exp. Parasitol. 48:81-91. The macrophages were maintained in DMEM medium at 37° C. and 5% CO_{2.} After 2 days in culture macrophages were challenged with a 10-fold excess of PNA—promastigotes for two hours. The macrophages were extensively washed with medium and incubated for 5 more days. Hoechst dye 33342 (Molecular Probes, Inc.) was then added to the cultures at a final concentration of 10 μg/ml. Fluorescence microscopy was carried out with an Olympus AX70 fluorescence microscope, and images were captured with a cooled CCD camera.

We observed green fluorescent parasites within the infected macrophages (FIG. 8). Counterstaining with Hoechst dye 33342 allowed us to assign the amastigotes nuclear and kinetoplast fluorescence to the green fluorescence within the macrophage. Interestingly, amastigotes of L. major strain SSU::IR1SAT-GFPa displayed a brighter fluorescence than members of the strain SSU::IR1SAT-GFPb. This is contrary to the situation in promastigotes and can be explained by the different, stage-dependent processing rates of RNA mediated by the IRs flanking the GFP gene. The 3′ UTR of GFP in expression site b is the L. donovani LPG1 IR and LPG biosynthesis is known to be downregulated in amastigotes.

Isolation of amastigote Leishmania from lesions Female 5-6 week old mice (Balb/c) were inoculated with 5×10⁶ PNA—promastigotes of the respective Leishmania strains. The parasites were injected into the footpad of the right hind leg. After 3 weeks amastigote Leishmania were isolated from non-necrotic lesions by subsequent filtration of homogenized tissue through polycarbonate filters of decreasing pore size as described by Glaser et al. (1990) Exper. Parasitol. 71:343-345.

As in the infected macrophages in culture, lesion-derived amastigotes of strain SSU::IR1SAT-GFPa were brighter than SSU::IR1SAT-GFPb amastigotes. These data confirm that the amastigotes display a fluorescence higher than the stationary metcyclic relatives. The intensity of L. major SSU::IR1SAT-GFPa amastigotes is about twice as high as that of PXG-GFP+ transfected promastigotes.

These examples demonstrate using the GFP that heterologous genes which utilize the rRNA promoter are highly expressed in promastigote and amastigote stages of the parasite. Expression of integrated GFP genes reflects the transcriptional activity within the ribosomal locus as driven by the ribosomal promotor and thus expression of heterologous genes is dependent on the proliferation status of the parasite. In addition, the UTRs used to assure co—and posttranscriptional processing of the RNA have a pronounced effect on absolute expression levels.

The green fluorescent cell lines which are easy to detect are a useful tool to study Leishmania virulence and pathogenicity. For example, the fate of a single parasite can be followed during in vitro infection experiments with isolated macrophages. Questions of organ tropism can be answered or colonization kinetics of mammalian hosts followed much more readily than before. Furthermore, the immediate monitoring of transcriptional activity within the ribosomal locus provides an opportunity to use these cell lines as reporters searching for cis and trans activating factors regulating RNA polymerase I transcription.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

Appendix 1. Sequence information

SEQ ID NO:1 Forward primer for amplifying conserved region of SSU rDNA—(SMB600)

5 5′-ggccaatatttaaattggataacttggcg-3′

SEQ ID NO:2 Reverse primer for above (SMB601)

5′-ccggaatatttaaatatcggtgaactttcgg-3′

SEQ ID NO:3 PIR1-SAT

LOCUS pIR1SAT 8493 bp DNA CIRCULAR SYN

24-MAR-1999

DEFINITION pIR1-SAT

ACCESSION pIR1SAT

KEYWORDS

SOURCE Unknown.

ORGANISM Leishmania sp.

Order Kinetoplastida, Family Trypanosomatidae

REFERENCE 1 (bases 1 to 8493)

AUTHORS S. M. Beverley, Washington University School of Medicine

JOURNAL Unpublished.

FEATURES Location/Qualifiers

CDS

1 . . . 913

/gene=″L. major SSU′″

/product=″Leishmania major SSU, 5′ part ″

/corresponds to nucleotides 123-1035 of GenBank X53915

MISC

942 . . . 1179

/region=″DST IR″

/Leishmania major intergenic region 5′ of DST gene

/corresponds to nucleotides 3816-4053 of GenBank X51733

MISC

1204 . . . 2532

/region=″CYS2 IR″

/Leishmania pifanoi intergenic region 5′ of CYS2 gene

/contains nucleotides 1501-2662, 1-167 of GenBank M97695

MISC

2795 . . . 3343

/region=″LPG1 IR″

/Leishmania donovani intergenic region 5′ of LPG1 gene

/contains nucleotides 1420-1969 of GenBank L11348

CDS

3401 . . . 3927

/gene=″SAT″

/product=″streptothricin acetyltransferase″

/corresponds to nucleotides 257-783 of GenBank X15995

MISC

3978 . . . 4549

/region=″1.7K IR″

/Leishmania major intergenic region 5′ of 1.7K mRNA

/corresponds to nucleotides 6-577 of GenBank X51734

CDS

4546 . . . 5631

/gene=″L. major ′SSU″

/product=″Leishmania major SSU, 3′ part ″

/corresponds to nucleotides 1035-2119 of GenBank X53915

MISC

5632 . . . 8493

/region=bacterial vector

/modified PBSII SK-

CDS

complement (6848 . . . 7708)

/gene=″amp″

/product=″beta-lactamase″

BASE COUNT 1819 a 2333 c 2215 g 2126 t ORIGIN

1 AAATTGGATA ACTTGGCGAA ACGCCAAGCT AATACATGAA CCAACCGGGT GTTCTCCACT

61 CCAGACGGTG GGCAACCATC GTCGTGAGAC GCCCAGCGAA TGAATGACAG TAAAACCAAT

121 GCCTTCACTG GCAGTAACAC CCAGCAGTGT TGACTCAATT CATTCCGTGC GAAAGCCGGC

181 TTGTTCCGGC GTCTTTTGAC GAACAACTGC CCTATCAGCT GGTGATGGCC GTGTAGTGGA

241 CTGCCATGGC GTTGACGGGA GCGGGGGATT AGGGTTCGAT TCCGGAGAGG GAGCCTGAGA

301 AATAGCTACC ACTTCTACGG AGGGCAGCAG GCGCGCAAAT TGCCCAATGT CAAAACAAAA

361 CGATGAGGCA GCGAAAAGAA ATAGAGTTGT CAGTCCATTT GGATTGTCAT TTCAATGGGG

421 GATATTTAAA CCCATCCAAT ATCGAGTAAC AATTGGAGGA CAAGTCTGGT GCCAGCACCC

481 GCGGTAATTC CAGCTCCAAA AGCGTATATT AATGCTGTTG CTGTTAAAGG GTTCGTAGTT

541 GAACTGTGGG CTGTGCAGGT TTGTTCCTGG TCGTCCCGTC CATGTCGGAT TTGGTGACCC

601 AGGCCCTTGC AGCCCGTGAA CATTCAAAGA AACAAGAAAC ACGGGAGTGG TTCCTTTCCT

661 GATTTACGCA TGTCATGCAT GCCAGGGGGC GTCCGTGATT TTTTACTGTG ACTAAAGAAG

721 CGTGACTAAA GCAGTCATTT GACTTGAATT AGAAAGCATG GGATAACAAA GGAGCAGCCT

781 CTAGGCTACC GTTTCGGCTT TTGTTGGTTT TAAAGGTCTA TTGGAGATTA TGGAGCTGTG

841 CGACAAGTGC TTTCCCATCG CAACTTCGGT TCGGTGTGTG GCGCCTTTGA GGGGTTTAGT

901 GCGTCCGGTG CGATAGGGAG ACCACAACGG TTTCCCTCTA GTGCGTGAAG GGTTACCGCA

961 ACGATGCGCA ATGGACTCCC CCGCTTTCCA TTTCGTCACC TTCCGCCTCT CTCTCTCTCT

1021 CTCTCACCAT CTACGCGTGC ACATCATCAA CTGTCTCTTG TCGGTGCTCA CCACCCTCAA

1081 CCACCCCTCA CTTTCAAGGC TTCCCGAACG CACACAAAAG GCGTGAAAAC CGCTCGCGTG

1141 TGTTGAGCCG TCCACCGTAG CCCTCCCCCT GTCCCCGGGG GATCCACTAG TTCTAGAGGA

1201 TCGGAGGTGT GTGTGCCCTT GTGTGCTGTG TGTGGGTGGA CGCAGCGATG CCCGGCGCGT

1261 GTGGGCACCT CCTTGGGTGC GCGCCCGCCG TGGCAGCTGC GCGTGCGTGC GAGATGTGAG

1321 GCAGAGGAAG AGGAAGGCGA TGCGGGCGAC ACGCAGAGGT GCGGCGGACG TAGGGGGGAA

1381 ATGGACGAGC AGGCGCGCTG TGAATCGGAG CTGCGGCACC ACCCAAGTCG TGGTGCCCCG

1441 CGAATGGCTG TTCTGCCGCC CTCGCTTCAC GCCTCCCCCT CCCCTCGCGT GCCCTCGCGT

1501 GGCCTCCCTT GTTATCCCTC TCTCTCGCAC GCACACGGAT ACGCGAGCCC GCTATTCTGC

1561 CTTCGTCTGG CTCTTTGTAT TCTGCTTGCT TCTTCAGCAC ACTTGTGTGC TGTGCGTTCA

1621 GCGATATCTT CCACTACTTT GTTTTCTCCT CCCCCTCGGG AGGTGCTTCG CTTGTGCTTT

1681 GACGGTGGTG CGTGGCTGCT GGGTCATGTG CCGGGCGTGC GCGCCTCCGC CGCCTCCCTG

1741 CAGCTTGTGG GTCTGGCTGC GTTCGCACCG CGCTCGCGTG CATGCACATG CCTGCACTGC

1801 GTCGGGAACG ACTTCCGGGC GCGTTGGCCC CCCGCCTCTG CAGCCACGGT CTGTTTATTG

1861 ATTGTGCTTG CTTCATCGGC TCTTCTCTGC GCGCGTGCGT GCGTGCGTGT GCGTGTCCGT

1921 GCGTATGCGT GAGGCGCAAC GGTCCCCAGA GCAAGGCATG TCGAGGGGAA CACTATAGAC

1981 GCATGTGTAC GTGTACACGA TGTGTATACG TATACGTGTA CCGAATGGTG CGTGCGCGTG

2041 TGCAGCATTG CCGTGACGGC ATGTACGAAG CGCTGCAGTG GGATGGACCC TGTGCGCGTG

2101 CCGGAGAGGT AGTGTCGCGT GTGGGTGCGG AGTGATGGAG GCTAGGGGGC TTACGAGCAC

2161 CGTCGCTTTT CCCCCGATGG CGGCTGGCAC GCAGCGCACG CACCGGGGAT GTGTGACGTG

2221 CGTCCTGTGC GCCTCTCCCT CTCCCCTTGT CGCCGGCGCA TGGATGCACC GCTGTTGTGT

2281 GAGGTTGCCC GCACCTGCGT TGTTGCCTGT GATGACGTCC CTCCCTCTCT TGCACTCTCC

2341 CCGTCCCCAC CTGCCCTGCA CCGTGGTCGA CTGCTCCCGA CGCCCTGCAC AGACTCTCGT

2401 CGCCACCACC AGCAGCAGCC CTCTATATAC CCGCCACTGC CGTAGCGTTC GGGCCGTGGC

2461 TCTGCGTTTC ACTTGCTCTC CCCTCGCTCT GTTCATTGCT TCCTTCTGTT CCCCTCGCTG

2521 CCCGCGTCCG GAGATCTATG AGTCTTGTGA TGTACTGGCT GATTTCTACG ACCAGTTCGC

2581 TGACCAGTTG CACGAGTCTC AATTGGACAA AATGCCAGCA CTTCCGGCTA AAGGTAACTT

2641 GAACCTCCGT GACATCTTAG AGTCGGACTT CGCGTTCGCG TAACGCCAAA TCAATACGAC

2701 CCGGATCTCC CTTTAGTGAG GGTTAATTAG TCCTGCATTA ATGAATCGGC CAACGCGCGG

2761 GGAGAGGCGG TTTGCGTATT GGGCGCTCTT CCGCTACTCG GGTGTCGCAC ACACTGTAAA

2821 ACGCCCCCGC CGGCTCTGTC ACGCAAGAAA CGAGAGCAAA AAGACCGGTA GACTATATCA

2881 CGCACAATCA CCGCGTGTGC GTCTCCCTGG GTGAAGACAC CCATCGCACC CTTCGACAGC

2941 CGCCCTTATG CCTATTCACC GTCTGTAGAA CACACAAGAG GAATAGCCCG GTGCCGCGTG

3001 CAAGACTGCG GCTTCTGCAC GCACTATGCT CGTTTCCGCC TCTCTCTCTT TGTGCGCGTG

3061 TGTGTGTGTG TGTCGGAGTG GCCCTCCCGT TACGTCTTTT GGGGGTGGGT GATAGCGGCA

3121 GATGCTGCTT CGACCTTGTG CGCCGCACCG GTGCCGTTGG CTACACTGCG GAAGGCAACA

3181 CAGAACACAC CCTGTGCCAT TTCTTCTTTT TTTTTTGCTT TCACCCACCT TTTCCCCGTG

3241 CTTCCCCATC TTTCCCCCTC TTTCCCTAAC GTACATTGCA CCTCTCCTTA TCGTGCAGTC

3301 ACACGCTACC ACTCAACGCT CCCTGCAACA CTGGAGTGAG TCGCTAGAAA TAATTTTGTT

3361 TAACTTTAAG AAGGAGATAT ACATAGTGAC CGGATCCTAG TATGAAGATT TCGGTGATCC

3421 CTGAGCAGGT GGCGGAAACA TTGGATGCTG AGAACCATTT CATTGTTCGT GAAGTGTTCG

3481 ATGTGCACCT ATCCGACCAA GGCTTTGAAC TATCTACCAG AAGTGTGAGC CCCTACCGGA

3541 AGGATTACAT CTCGGATGAT GACTCTGATG AAGACTCTGC TTGCTATGGC GCATTCATCG

3601 ACCAAGAGCT TGTCGGGAAG ATTGAACTCA ACTCAACATG GAACGATCTA GCCTCTATCG

3661 AACACATTGT TGTGTCGCAC ACGCACCGAG GCAAAGGAGT CGCGCACAGT CTCATCGAAT

3721 TTGCGAAAAA GTGGGCACTA AGCAGACAGC TCCTTGGCAT ACGATTAGAG ACACAAACGA

3781 ACAATGTACC TGCCTGCAAT TTGTACGCAA AATGTGGCTT TACTCTCGGC GGCATTGACC

3841 TGTTCACGTA TAAAACTAGA CCTCAAGTCT CGAACGAAAC AGCGATGTAC TGGTACTGGT

3901 TCTCGGGAGC ACAGGATGAC GCCTAACTAG CCTCGGAGAT CCACTAGTTC TAGTTCTAGG

3961 GGGCGCGAAT TCAGATCCTC GTGTGAGCGT TCGCGGAATC GGTCGCTCGT GTTTATGCCC

4021 GTCTTGGTGT TGTGCTCGCA AGGCGGTGCA GCAGGATACC GTCGCCCTCC TCTCTCCTTG

4081 CTTCTCTGTT CTTCAATTCG CGATCTCACA GAGGCCGGCT GTGCACGCCC TTCCTCACCC

4141 TCCTTTTCCC ACCTCTCGGC CACCGGTCGG CTCCGTTCCG TCTGCCGTCG AGAAGGGACG

4201 GGCATGTGCA GCTCCTCCCT TTCTCTCGCG CGCGCATCTT CTCTTGCTTG TGGCACTCAC

4261 GCTCATGCGT CAAGGCGGCC CCACGCGAGC CCCTGCGCTC CCTTCCCTCT TGCGCATCCG

4321 TAGCCGGACT GGTCGATGCG CAAGGCCGGC ATGAAGGAGC GCGTGCCCTC AAGAGCGGAC

4381 TATCATGCCC TACGTGGGCC ACGCAGCGAT GAGGCCGGCT TCGCGGAGAT GCGTCACGCA

4441 CGTGCCAGAT GATGCCGTAC GCCTTCCTTG ACTTGCGCCC CCCTCTCTTC CTCCGTCTCT

4501 CACTCTCTCT CTCTCACACA CACACACACA CACACACACA CACAAAGCTC CGGTTCGTCC

4561 GGCCGTAACG CCTTTTCAAC TCACGGCCTC TAGGAATGAA GGAGGGTAGT TCGGGGGAGA

4621 ACGTACTGGG GCGTCAGAGG TGAAATTCTT AGACCGCACC AAGACGAACT ACAGCGAAGG

4681 CATTCTTCAA GGATACCTTC CTCAATCAAG AACCAAAGTG TGGAGATCGA AGATGATTAG

4741 AGACCATTGT AGTCCACACT GCAAACGATG ACACCCATGA ATTGGGGATC TTATGGGCCG

4801 GCCTGCGGCA GGGTTTACCC TGTGTCAGCA CCGCGCCCGC TTTTACCAAC TTACGTATCT

4861 TTTCTATTCG GCCTTTACCG GCCACCCACG GGAATATCCT CAGCACGTTT TCTGTTTTTT

4921 CACGCGAAAG CTTTGAGGTT ACAGTCTCAG GGGGGAGTAC GTTCGCAAGA GTGAAACTTA

4981 AAGAAATTGA CGGAATGGCA CCACAAGACG TGGAGCGTGC GGTTTAATTT GACTCAACAC

5041 GGGGAACTTT ACCAGATCCG GACAGGATGA GGATTGACAG ATTGAGTGTT CTTTCTCGAT

5101 TCCCTGAATG GTGGTGCATG GCCGCTTTTG GTCGGTGGAG TGATTTGTTT GGTTGATTCC

5161 GTCAACGGAC GAGATCCAAG CTGCCCAGTA GAATTCAGAA TTGCCCATAG AATAGCAAAC

5221 TCATCGGCGG GTTTTACCCA ACGGTGGGCC GCATTCGGTC GAATTCTTCT CTGCGGGATT

5281 CCTTTGTAAT TGCACAAGGT GAAATTTTGG GCAACAGCAG GTCTGTGATG CTCCTCAATG

5341 TTCTGGGCGA CACGCGCACT ACAATGTCAG TGAGAACAAG AAAAACGACT TTTGTCGAAC

5401 CTACTTGATC AAAAGAGTGG GGAAACCCCG GAATCACATA GACCCACTTG GGACCGAGGA

5461 TTGCAATTAT TGGTCGCGCA ACGAGGAATG TCTCGTAGGC GCAGCTCATC AAACTGTGCC

5521 GATTACGTCC CTGCCATTTG TACACACCGC CCGTCGTTGT TTCCGATGAT GGTGCAATAC

5581 AGGTGATCGG ACAGGCGGTG TTTTATCCGC CCGAAAGTTC ACCGATATTT AAATCCAGCT

5641 TTTGTTCCCT TTAGTGAGGG TTAATTGCGC GCTTGGCGTA ATCATGGTCA TAGCTGTTTC

5701 CTGTGTGAAA TTGTTATCCG CTCACAATTC CACACAACAT ACGAGCCGGA AGCATAAAGT

5761 GTAAAGCCTG GGGTGCCTAA TGAGTGAGCT AACTCACATT AATTGCGTTG CGCTCACTGC

5821 CCGCTTTCCA GTCGGGAAAC CTGTCGTGCC AGCTGCATTA ATGAATCGGC CAACGCGCGG

5881 GGAGAGGCGG TTTGCGTATT GGGCGCTCTT CCGCTTCCTC GCTCACTGAC TCGCTGCGCT

5941 CGGTCGTTCG GCTGCGGCGA GCGGTATCAG CTCACTCAAA GGCGGTAATA CGGTTATCCA

6001 CAGAATCAGG GGATAACGCA GGAAAGAACA TGTGAGCAAA AGGCCAGCAA AAGGCCAGGA

6061 ACCGTAAAAA GGCCGCGTTG CTGGCGTTTT TCCATAGGCT CCGCCCCCCT GACGAGCATC

6121 ACAAAAATCG ACGCTCAAGT CAGAGGTGGC GAAACCCGAC AGGACTATAA AGATACCAGG

6181 CGTTTCCCCC TGGAAGCTCC CTCGTGCGCT CTCCTGTTCC GACCCTGCCG CTTACCGGAT

6241 ACCTGTCCGC CTTTCTCCCT TCGGGAAGCG TGGCGCTTTC TCATAGCTCA CGCTGTAGGT

6301 ATCTCAGTTC GGTGTAGGTC GTTCGCTCCA AGCTGGGCTG TGTGCACGAA CCCCCCGTTC

6361 AGCCCGACCG CTGCGCCTTA TCCGGTAACT ATCGTCTTGA GTCCAACCCG GTAAGACACG

6421 ACTTATCGCC ACTGGCAGCA GCCACTGGTA ACAGGATTAG CAGAGCGAGG TATGTAGGCG

6481 GTGCTACAGA GTTCTTGAAG TGGTGGCCTA ACTACGGCTA CACTAGAAGG ACAGTATTTG

6541 GTATCTGCGC TCTGCTGAAG CCAGTTACCT TCGGAAAAAG AGTTGGTAGC TCTTGATCCG

6601 GCAAACAAAC CACCGCTGGT AGCGGTGGTT TTTTTGTTTG CAAGCAGCAG ATTACGCGCA

6661 GAAAAAAAGG ATCTCAAGAA GATCCTTTGA TCTTTTCTAC GGGGTCTGAC GCTCAGTGGA

6721 ACGAAAACTC ACGTTAAGGG ATTTTGGTCA TGAGATTATC AAAAAGGATC TTCACCTAGA

6781 TCCTTTTAAA TTAAAAATGA AGTTTTAAAT CAATCTAAAG TATATATGAG TAAACTTGGT

6841 CTGACAGTTA CCAATGCTTA ATCAGTGAGG CACCTATCTC AGCGATCTGT CTATTTCGTT

6901 CATCCATAGT TGCCTGACTC CCCGTCGTGT AGATAACTAC GATACGGGAG GGCTTACCAT

6961 CTGGCCCCAG TGCTGCAATG ATACCGCGAG ACCCACGCTC ACCGGCTCCA GATTTATCAG

7021 CAATAAACCA GCCAGCCGGA AGGGCCGAGC GCAGAAGTGG TCCTGCAACT TTATCCGCCT

7081 CCATCCAGTC TATTAATTGT TGCCGGGAAG CTAGAGTAAG TAGTTCGCCA GTTAATAGTT

7141 TGCGCAACGT TGTTGCCATT GCTACAGGCA TCGTGGTGTC ACGCTCGTCG TTTGGTATGG

7201 CTTCATTCAG CTCCGGTTCC CAACGATCAA GGCGAGTTAC ATGATCCCCC ATGTTGTGCA

7261 AAAAAGCGGT TAGCTCCTTC GGTCCTCCGA TCGTTGTCAG AAGTAAGTTG GCCGCAGTGT

7321 TATCACTCAT GGTTATGGCA GCACTGCATA ATTCTCTTAC TGTCATGCCA TCCGTAAGAT

7381 GCTTTTCTGT GACTGGTGAG TACTCAACCA AGTCATTCTG AGAATAGTGT ATGCGGCGAC

7441 CGAGTTGCTC TTGCCCGGCG TCAATACGGG ATAATACCGC GCCACATAGC AGAACTTTAA

7501 AAGTGCTCAT CATTGGAAAA CGTTCTTCGG GGCGAAAACT CTCAAGGATC TTACCGCTGT

7561 TGAGATCCAG TTCGATGTAA CCCACTCGTG CACCCAACTG ATCTTCAGCA TCTTTTACTT

7621 TCACCAGCGT TTCTGGGTGA GCAAAAACAG GAAGGCAAAA TGCCGCAAAA AAGGGAATAA

7681 GGGCGACACG GAAATGTTGA ATACTCATAC TCTTCCTTTT TCAATATTAT TGAAGCATTT

7741 ATCAGGGTTA TTGTCTCATG AGCGGATACA TATTTGAATG TATTTAGAAA AATAAACAAA

7801 TAGGGGTTCC GCGCACATTT CCCCGAAAAG TGCCACCTGA CGCGCCCTGT AGCGGCGCAT

7861 TAAGCGCGGC GGGTGTGGTG GTTACGCGCA GCGTGACCGC TACACTTGCC AGCGCCCTAG

7921 CGCCCGCTCC TTTCGCTTTC TTCCCTTCCT TTCTCGCCAC GTTCGCCGGC TTTCCCCGTC

7981 AAGCTCTAAA TCGGGGGCTC CCTTTAGGGT TCCGATTTAG TGCTTTACGG CACCTCGACC

8041 CCAAAAAACT TGATTAGGGT GATGGTTCAC GTAGTGGGCC ATCGCCCTGA TAGACGGTTT

8101 TTCGCCCTTT GACGTTGGAG TCCACGTTCT TTAATAGTGG ACTCTTGTTC CAAACTGGAA

8161 CAACACTCAA CCCTATCTCG GTCTATTCTT TTGATTTATA AGGGATTTTG CCGATTTCGG

8221 CCTATTGGTT AAAAAATGAG CTGATTTAAC AAAAATTTAA CGCGAATTTT AACAAAATAT

8281 TAACGCTTAC AATTTCCATT CGCCATTCAG GCTGCGCAAC TGTTGGGAAG GGCGATCGGT

8341 GCGGGCCTCT TCGCTATTAC GCCAGCTGGC GAAAGGGGGA TGTGCTGCAA GGCGATTAAG

8401 TTGGGTAACG CCAGGGTTTT CCCAGTCACG ACGTTGTAAA ACGACGGCCA GTGAGCGCGC

8461 GTAATACGAC TCACTATAGG GCGAATTGGA TTT / /

What is claimed is:

1. An expression cassette comprising
(a) flanking regions which are homologous to a region of a ribosomal RNA gene from an organism selected from the group consisting of Leishmania spp., Crithidia spp. or Leptomonas spp.;
(b) intergenic regions which contain information required for RNA transcript processing in the organism; and
(c) a marker gene operably linked to the intergenic regions which allows selection of individuals of the organism which are transfected with the DNA molecule.

2. The expression cassette of claim 1, wherein the region of a ribosomal RNA gene is a conserved region of the small subunit of the ribosomal RNA gene of a Leshmania sp.

3. The expression cassette of claim 1, consisting essentially of the larger fragment resulting from a Swa1 digest of pIR1-SAT.

4. The expression cassette of claim 1, further comprising a second gene encoding a protein, wherein the second gene is operably linked to the intergenic regions.

5. The expression cassette of claim 4, wherein the second gene encodes a protein selected from the group consisting of a green fluorescent protein, insulin, γ-interferon, tissue plasminogen activator, β-interferon, erythropoietin, Factor VIII, and a protein which is deficient or inactive in a lysosomal storage disease.

6. The expression cassette of claim 4, consisting essentially of the larger fragment resulting from a Swa1 digest of pIR1-SAT, and the second gene.

7. The expression cassette of claim 5, wherein the second gene encodes the green fluorescent protein.

8. An expression cassette comprising
(a) flanking regions which are homologous to a conserved region of the small subunit ribosomal RNA gene from an organism which undergoes trans-splicing;
(b) intergenic regions which contain information required for RNA transcript processing in the organism; and
(c) a marker gene operably linked to the intergenic regions which allows selection of individuals of the organism which are transfected with the DNA molecule.

9. The expression cassette of claim 8, wherein the organism is selected from the group consisting of Trypanosoma spp., Leishmania spp., Crithidia spp. and Leptomonas spp.

10. The expression cassette of claim 8, further comprising a second gene encoding a protein, wherein the second gene is operably linked to the intergenic regions.

11. The expression cassette of claim 10, wherein the organism is selected from the group consisting of Trypanosoma spp., Leishmania spp., Crithidia spp. or Leptomonas spp.

12. The expression cassette of claim 10, wherein the protein is selected from the group consisting of a green fluorescent protein, insulin, γ-interferon, tissue plasminogen activator, β-interferon, erythropoietin, Factor VIII, and a protein which is deficient or inactive in a lysosomal storage disease.

13. The expression cassette of claim 12, wherein the protein is the green fluorescent protein.

14. An expression cassette comprising
(a) a promoter for a ribosomal RNA gene from an organism which undergoes trans-splicing;
(b) flanking sequences which are homologous to a chromosomal region of the organism;
(c) intergenic regions which contain information required for RNA transcript processing in the organism; and
(d) a marker gene operably linked to the intergenic regions which allows selection of individuals of the organism which are transfected with the DNA molecule.

15. The expression cassette of claim 14, wherein the organism is selected from the group consisting of Trypanosoma spp., Leishmania spp., Crithidia spp. and Leptomonas spp.

16. The expression cassette of claim 14, further comprising a second gene encoding a protein, wherein the second gene is operably linked to the intergenic regions.

17. The expression cassette of claim 16, wherein the protein is selected from the group consisting of a green fluorescent protein, insulin, γ-interferon, tissue plasminogen activator, β-interferon, erythropoietin, Factor VIII, and a protein which is deficient or inactive in a lysosomal storage disease.

18. A recombinant plasmid comprising the expression cassette of claim 1 and DNA sequences which allow selection and replication of the vector in E. coli.

19. The recombinant plasmid of claim 18, consisting essentially of pIR1-SAT.

20. A recombinant plasmid comprising the expression cassette of claim 4 and DNA sequences which allow selection and replication of the vector in E. coli.

21. The recombinant plasmid of claim 20, wherein the second gene encodes a protein selected from the group consisting of a green fluorescent protein, insulin, γ-interferon, tissue plasminogen activator, β-interferon, erythropoietin, Factor VIII, and a protein which is deficient or inactive in a lysosomal storage disease.

22. A recombinant plasmid comprising the expression cassette of claim 8 and DNA sequences which allow selection and replication of the vector in E. coli.

23. A recombinant plasmid comprising the expression cassette of claim 10 and DNA sequences which allow selection and replication of the vector in E. coli.

24. A recombinant plasmid comprising the expression cassette of claim 14 and DNA sequences which allow selection and replication of the vector in E. coli.

25. A recombinant plasmid comprising the expression cassette of claim 16 and DNA sequences which allow selection and replication of the vector in E. coli.

26. A host cell of an organism which undergoes trans-splicing transformed with the expression cassette of claim 4, wherein said host cell comprises a chromosome.

27. The host cell of claim 26, wherein the expression cassette is integrated into the chromosome.

28. The host cell of claim 27, wherein the organism is Leishmania tarentolae.

29. The host cell of claim 27, wherein the second gene encodes a protein selected from the group consisting of a green fluorescent protein, insulin, γ-interferon, tissue plasminogen activator, β-interferon, erythropoietin, Factor VIII, and a protein which is deficient or inactive in a lysosomal storage disease.

30. The host cell of claim 27, wherein the second gene encodes a green fluorescent protein.

31. A host cell of an organism which undergoes trans-splicing transformed with the expression cassette of claim 10, wherein said host cell comprises a chromosome.

32. The host cell of claim 31, wherein the expression cassette is integrated into the chromosome.

33. The host cell of claim 32, wherein the organism is selected from the group consisting of Trypanosoma spp., Leishmania spp., Crithidia spp. and Leptomonas spp.

34. The host cell of claim 32, wherein the protein is selected from the group consisting of a green fluorescent protein, insulin, β-interferon, tissue plasminogen activator, β-interferon, erythropoietin, Factor VIII, and a protein which is deficient or inactive in a lysosomal storage disease.

35. A host cell of an organism which undergoes trans-splicing transformed with the expression cassette of claim 16, wherein said host cell comprises a chromosome.

36. The host cell of claim 35, wherein the expression cassette is integrated into the chromosome.

37. The host cell of claim 36, wherein the protein is selected from the group consisting of a green fluorescent protein, insulin, β-interferon, tissue plasminogen activator, β-interferon, erythropoietin, Factor VIII, and a protein which is deficient or inactive in a lysosomal storage disease.

38. A method of producing a protein, comprising:
(a) obtaining the host cell of claim 27, wherein the host cell further comprises cellular components, and
(b) culturing the host cell under conditions and for a time sufficient to produce the protein.

39. The method of claim 38, further comprising:
separating the protein from the cellular components.

40. The method of claim 38, wherein the protein is selected from the group consisting of a green fluorescent protein, insulin, β-interferon, tissue plasminogen activator, β-interferon, erythropoietin, Factor VIII, and a protein which is deficient or inactive in a lysosomal storage disease.

41. A method of producing a protein, comprising:
(a) obtaining the host cell of claim 32, wherein the host cell further comprises cellular components, and
(b) culturing the host cell under conditions and for a time sufficient to produce the protein.

42. The method of claim 41, further comprising:
separating the protein from the cellular components.

43. The method of claim 41, wherein the organism is selected from the group consisting of Trypanosoma spp., Leishmania spp., Crithidia spp. and Leptomonas spp.

44. The method of claim 41, wherein the protein is selected from the group consisting of a green fluorescent protein, insulin, γ-interferon, tissue plasminogen activator, β-interferon, erythropoietin, Factor VIII, and a protein which is deficient or inactive in a lysosomal storage disease.

45. A method of producing a protein, comprising:
(a) obtaining the host cell of claim 36, wherein the host cell further comprises cellular components, and
(b) culturing the host cell under conditions and for a time sufficient to produce the protein.

46. The method of claim 45, further comprising:
separating the protein from the cellular components.

47. The method of claim 45, wherein the organism is selected from the group consisting of Trypanosoma spp., Leishmania spp., Crithidia spp. and Leptomonas spp.

48. The method of claim 45, wherein the protein is selected from the group consisting of a green fluorescent protein, insulin, β-interferon, tissue plasminogen activator, β-interferon, erythropoietin, Factor VIII, and a protein which is deficient or inactive in a lysosomal storage disease.

49. A method for studying virulence or pathogenicity in a trans-splicing organism, comprising infecting an experimental animal with the recombinant host cell of claim 27, wherein the protein is a green fluorescent protein.

50. A method for studying virulence or pathogenicity in a trans-splicing organism, comprising infecting an experimental animal with the recombinant host cell of claim 32, wherein the protein is a green fluorescent protein.

51. A method for studying virulence or pathogenicity in a trans-splicing organism, comprising infecting an experimental animal with the recombinant host cell of claim 36, wherein the protein is a green fluorescent protein.

52. A method of treating a disease or undesirable condition in a mammal, comprising infecting the mammal with an infectious strain of the host cell of claim 27, wherein the protein is useful for treating the disease or undesirable condition.

53. The method of claim 52, wherein the mammal is a human and the disease or undesirable condition is selected from the group consisting of osteoporosis, diabetes, cancer, severe anemia, short stature, hemophilia, and lysosomal storage diseases.

54. The method of claim 53, wherein the disease or undesirable condition is Goucher Disease or Fabry Disease.

55. A method of treating a disease or undesirable condition in a mammal, comprising infecting the mammal with an infectious strain of the host cell of claim 32, wherein the protein is useful for treating the disease or undesirable condition.

56. The method of claim 55, wherein the mammal is a human and the disease or undesirable condition is selected from the group consisting of osteoporosis, diabetes, cancer, severe anemia, short stature, hemophilia, and lysosomal storage diseases.

57. The method of claim 56, wherein the disease or undesirable condition is Goucher Disease or Fabry Disease.

58. A method of treating a disease or undesirable condition in a mammal, comprising infecting the mammal with an infectious strain of the host cell of claim 36, wherein the protein is useful for treating the disease or undesirable condition.

59. The method of claim 58, wherein the mammal is a human and the disease or undesirable condition is selected from the group consisting of osteoporosis, diabetes, cancer, severe anemia, short stature, hemophilia, and lysosomal storage diseases.

60. The method of claim 59, wherein the disease Goucher Disease or Fabry Disease.

61. A method of delivering a therapeutic protein to a desired site in a mammal, comprising
(a) selecting a trans-splicing organism which is capable of infecting the mammal and residing at the desired site;
(b) transfecting the trans-splicing organism with the expression cassette of claim 4, wherein the second gene encodes the therapeutic protein; and
(c) infecting the mammal with the transfected trans-splicing organism.

62. The method of claim 61, wherein the mammal is a human and the trans-splicing organism is selected from the group consisting of Leishmania spp. and Trypanosoma spp.

63. The method of claim 62, wherein the site is a lysosome and the trans-splicing organism is a Leishmania.

64. A method of delivering a therapeutic protein to a desired site in a mammal, comprising
(a) selecting a trans-splicing organism which is capable of infecting the mammal and residing at the desired site;
(b) transfecting the trans-splicing organism with the expression cassette of claim 10, wherein the second gene encodes the therapeutic protein; and
(c) infecting the mammal with the transfected trans-splicing organism.

65. The method of claim 64, wherein the mammal is a human and the trans-splicing organism is selected from the group consisting of Leishmania spp. and Trypanosoma spp.

66. The method of claim 65, wherein the site is a lysosome and the trans-splicing organism is a Leishmania.

67. A method of delivering a therapeutic protein to a desired site in a mammal, comprising
(a) selecting a trans-splicing organism which is capable of infecting the mammal and residing at the desired site;
(b) transfecting the trans-splicing organism with the expression cassette of claim 16, wherein the second gene encodes the therapeutic protein; and
(c) infecting the mammal with the transfected trans-splicing organism.

68. The method of claim 67, wherein the mammal is a human and the trans-splicing organism is selected from the group consisting of Leishmania spp. and Trypanosoma spp.

69. The method of claim 68, wherein the site is a lysosome and the trans-splicing organism is a Leishmania.

70. A kit for producing a recombinant protein, comprising the recombinant plasmid of claim 18, a living cell of the organism, and instructions.

71. The kit of claim 70, wherein the organism is Leishmania tarentolae.

72. A kit for producing a recombinant protein, comprising the recombinant plasmid of claim 22, a living cell of the organism, and instructions.

73. The kit of claim 72, wherein the organism is selected from the group consisting of Trypanosoma spp., Leishmania spp., Crithidia spp. and Leptomonas spp.

74. The kit of claim 73, wherein the recombinant plasmid is pIR1SAT.

75. The kit of claim 72, wherein the organism is selected from the group consisting of Crithidia spp., Leptomonas spp., and Leishmania tarentolae.

76. A kit for producing a recombinant protein, comprising the recombinant plasmid of claim 24, a living cell of the organism, and instructions.

77. The kit of claim 76, wherein the organism is selected from the group consisting of Trypanosoma spp., Leishmania spp., Crithidia spp. and Leptomonas spp.

78. The kit of claim 76, wherein the organism is selected from the group consisting of Crithidia spp., Leptomonas spp., and Leishmania tarentolae.
